SEQUENCE LISTING (1 sequence)

SEQ ID NO: 1 - protein, 15 aa, Artificial Sequence
  Acceptor Substrate Peptide
LKNVPTPSPSPKPQH

(19) United States
(12) Patent Application Publication
(10) Pub. No.: US 2017/0218032 A1
(43) Pub. Date: Aug. 3, 2017

(54) YEAST O-MANNOSE NUCLEOCYTOPLASMIC GLYCOSYLATION

(71) Applicants: Adnan HALIM, Malmo (SE); Hiren J. JOSHI, Copenhagen (DK); Ida Signe Bohse LARSEN, Copenhagen (DK); Yanqui YUAN, Copenhagen (DK); Henrik CLAUSEN, Copenhagen (DK)

(72) Inventors: Adnan HALIM, Malmo (SE); Hiren J. JOSHI, Copenhagen (DK); Ida Signe Bohse LARSEN, Copenhagen (DK); Yanqui YUAN, Copenhagen (DK); Henrik CLAUSEN, Copenhagen (DK)

(21) Appl. No.: 15/367,135

(22) Filed: Dec. 1, 2016

Related U.S. Application Data
(60) Provisional application No. 62/262,341, filed on Dec. 2, 2015.

(51) Int. Cl. C07K 14/395; C07K 14/39; C12P 21/02

(52) U.S. Cl. CPC C07K 14/395; C12P 21/02; C07K 14/39

(57) ABSTRACT

The present disclosure provides, inter alia, compositions and methods for modulating and/or utilizing nucleocytoplasmic alpha-mannosyltransferase enzyme activity in yeast, e.g., to modulate the performance of the yeast in fermentation, respiration, bioproduction and/or bioprocessing. The disclosure also provides, inter alia, modified eukaryotic organisms comprising modulated nucleocytoplasmic alpha-mannosyltransferase enzyme activity, and uses of such modified eukaryotic organisms.

### CROSS REFERENCE TO RELATED APPLICATIONS

This application claims priority to U.S. Provisional Application No. 62/262,341, filed on Dec. 2, 2015, which application is incorporated by reference herein in its entirety.

### TECHNICAL FIELD OF THE INVENTION

The present invention relates to a nucleocytoplasmic type of O-glycosylation found in all yeast that serves to modulate cell physiology and metabolism. The O-glycosylation modification of nucleocytoplasmic proteins in yeast may be used to manipulate yeast for wide improvements in fermentation and bioprocessing uses.

### BACKGROUND OF THE INVENTION

All eukaryotic cells except yeast harbor a simple type of protein O-glycosylation designated O-GlcNAcylation in the cytosol and nucleus (Hart, Housley et al. 2007). O-GlcNAcylation is an essential and dynamic process involving addition and removal of a single GlcNAc at Ser and Thr residues of nuclear, cytoplasmic and mitochondrial proteins (Torres and Hart 1984, Holt and Hart 1986, Haltiwanger, Blomberg et al. 1992, Dong and Hart 1994). The transfer of GlcNAc to proteins is carried out by the O-GlcNAc transferase (OGT) using uridine diphosphate α-D-GlcNAc (UDP-GlcNAc) as donor substrate, and a second hydrolytic enzyme, O-GlcNAcase (OGA), is available to remove the GlcNAc monosaccharide in a regulated dynamic process (Haltiwanger, Blomberg et al. 1992, Dong and Hart 1994). The OGT/OGA enzyme pair was initially identified in mammals but subsequent work has demonstrated O-GlcNAcylation and the OGT/OGA enzymes in bacteria, filamentous fungi, plants and metazoans. O-GlcNAcylation is a widespread modification found on e.g. nucleoporins, transcription factors, kinases, cytoskeletal and chromatin proteins, it is involved in a plethora of biological processes and believed to play causal roles in diabetes, cancer, cardiovascular- and Alzheimer's disease (Hart, Slawson et al. 2011, Hardiville and Hart 2014, Bond and Hanover 2015). Sites of O-GlcNAcylation are often found at or in close proximity to protein phosphorylation sites, and the intricate interplay between both modifications is known to modulate many important processes in cells (Hu, Shimoji et al. 2010, Hardiville and Hart 2014).

O-GlcNAc metabolism is believed to serve as a nutrient-sensing pathway designated the hexosamine biosynthetic pathway (HBP). This pathway is sensitive to carbohydrate, amino acid, fatty acid and ATP levels. The UDP-GlcNAc substrate for OGT is also the final product of the HBP and therefore O-GlcNAc glycosylation ideal to reflect the nutritional state of the cell. Specifically, key components that feed into UDP-GlcNAc synthesis are derived from nutrients, such as the amino acid glutamine, acetyl-coenzyme A (acetyl-CoA), glucose and uridine. In this regard, the enzymes of the HBP join the signaling proteins AMP activated protein kinase (AMPK) and mammalian target of rapamycin (mTOR), which act as sensors of energy charge and amino acid levels, respectively. Owing to its nutrient dependence, the presence or absence of an O-GlcNAc modification on a protein can initiate a cascade that is intended to direct the cell to augment processes that are associated with nutrient conservation, such as a reduction in cell proliferation and attenuation of nutrient storage and cell growth. Exactly how a balance is achieved between the apparently competing functions of OGA and OGT to modulate highly specific spatiotemporal responses is not fully understood, but a likely possibility is that under certain conditions O-GlcNAcylation of proteins might be influenced in a global manner. Concurrently, a more fine-tuned mechanism might govern protein O-GlcNAcylation to ensure both global and local cellular response to changes in UDP-GlcNAc levels, which would be important both under conditions of very low or very high UDP-GlcNAc, or under conditions of UDP-GlcNAc homeostasis.

The only eukaryotic cell type where O-GlcNAcylation and identifiable orthologous OGT/OGA genes have not been identified is yeast. Yeast use primarily Ser and Thr for phosphorylation with tyrosine (Tyr) phosphorylation being utilized to an extremely low extent (Chi, Huttenhower et al. 2007), this is in contrast to other eukaryotic cells that phosphorylate all three residues for extensive and vital signalling. It is unknown how yeast accommodate the many biological functions O-GlcNAcylation have in higher eukaryotic cells despite the similarities in usage of phosphorylation as the primary cell signalling system.

### SUMMARY OF THE INVENTION

The present invention relates to a nuclear, cytoplasmic and mitochondrial (hereafter referred to as “nucleocytoplasmic”) type of O-glycosylation found in all yeast (e.g., in Saccharomyces cerevisiae and Schizosaccharomyces pombe). The nucleocytoplasmic O-glycosylation in yeast is composed of alpha-linked mannose (O-Man) residue(s) which may cycle dynamically on and off proteins to modulate cellular functions including, but not limited to, metabolism, enzyme activity, protein localization, phosphorylation and cell signalling.

The invention further relates to an alpha-mannosyltransferase enzyme activity found soluble in total lysates of yeast. The nucleocytoplasmic O-glycosylation in yeast is shown to be carried out by a GDP-Man: polypeptide alpha-mannosyltransferase that transfers mannose residues from GDP-Man to nucleocytoplasmic proteins. The enzyme activity can be detected and quantified using for example a peptide substrate.

In some embodiments, the present invention relates to methods to enhance and/or interfere with nucleocytoplasmic O-Man in yeast. In some embodiments, such methods comprise enhancing or interfering with nucleocytoplasmic O-Man in yeast to modulate the performance of the yeast in fermentation, respiration, bioproduction and/or bioprocessing.

Another object of the present invention relates to methods to modulate (e.g., enhance and/or interfere with) nucleocytoplasmic O-Man in yeast to modulate cellular processes including, but not limited to carbon, nitrogen and/or lipid metabolite synthesis.

Yet another object of the present invention relates to methods to enhance and/or interfere with nucleocytoplasmic O-Man in yeast to modulate cellular processes including, but not limited to protein, nucleotide, and/or fatty acid synthesis.

Yet another object of the present invention relates to methods to enhance and/or interfere with nucleocytoplasmic O-Man in yeast to modulate cellular signaling pathways including, but not limited to, phosphorylation, acetylation, ubiquitination and/or methylation.

In one embodiment a plurality of O-Man nucleocytoplasmic glycoproteins and O-Man glycosites in these are provided.

In another embodiment mutations in the plurality of O-Man glycosites in proteins are provided to interfere with the direct or indirect biological functions of O-Man glycosylation.

In yet another embodiment a method for modulating the O-Man glycosylation and biological functions by nutrient supply is provided.

In yet another embodiment an enzyme assay to measure nucleocytoplasmic O-Man enzyme activity in the yeast nucleocytoplasm is provided.

In yet another embodiment a screen for modulators of the nucleocytoplasmic O-Man enzyme activity is provided.

In yet another embodiment characteristics of inhibitors and activators of nucleocytoplasmic O-Man enzyme activity are provided.

An object of the present invention relates to modulating the O-Man nucleocytoplasmic glycosylation in yeast to improve performance in bioprocessing and use of yeast as production platform for recombinant biologics.

An aspect of the present invention relates to modulating the O-Man nucleocytoplasmic glycosylation in yeast to enhance or inhibit carbon metabolism.

In one embodiment the present invention relates to modulation of nucleocytoplasmic O-Man glycosylation found on carbohydrate transporters, including, but not limited to AGT1, GAL2, HXT1-17 and MAL61 to modulate intracellular uptake and/or downstream carbon metabolism.

In yet another embodiment the present invention relates to modulation of nucleocytoplasmic O-Man glycosylation found on enzymes catalyzing glycolysis and/or gluconeogenesis, including, but not limited to, HXK1, HXK2, GLK1, PGI1, PFK1, PFK2, FBP1, FBA1, TPI1, GPD1, GPD2, GUT2, GPP1, GPP2, GUT1, THD1-3, PGK1, GPM1, ENO1, ENO2, PYK1, PYK2, PDA1, PDB1, LAT1, LPD1, PDX1, PCK1, PYC1 and PYC2 to modulate carbon metabolism, growth on non-fermentable compounds and/or levels of downstream carbon metabolites.

In yet another embodiment the present invention relates to modulation of nucleocytoplasmic O-Man glycosylation found on enzymes catalyzing the pentose phosphate pathway, also known as the hexose monophosphate pathway, including, but not limited to, ZWF1, GND1, GND2, RPE1, RKI1, TLK1, TLK2 and TAL1 to modulate carbon metabolism and/or levels of downstream carbon metabolites.

In yet another embodiment the present invention relates to modulation of nucleocytoplasmic O-Man glycosylation found on enzymes catalyzing fermentation, including, but not limited to, PDC1, PDC5, ACS1, ACS2, PDC6, ALD4, ALD6 and ADH1-5, to modulate ethanol production.

In yet another embodiment the present invention relates to modulation of nucleocytoplasmic O-Man glycosylation found on enzymes catalyzing the citric acid cycle, also known as Krebs cycle, including, but not limited to, CIT1-3, ACO1, IDH1, IDH2, IDP1, IDP2, KGD1, KGD2, ACS1, ACS2, LPD1, LSC1, LSC2, SDH1-4, FUM1, MDH1-3, ICL1, MLS1 and MLS2 to modulate carbon metabolism and/or downstream carbon metabolites.

In yet another embodiment the present invention relates to modulation of nucleocytoplasmic O-Man glycosylation found on enzymes catalyzing glycolysis, gluconeogenesis, fermentation, pentose phosphate pathway and/or citric acid cycle to modulate nicotinamide adenine dinucleotide [NAD(H)], nicotinamide adenine dinucleotide phosphate [NADP(H)] and/or flavin adenine dinucleotide [FAD(H2)] levels.

In yet another embodiment the present invention relates to modulation of nucleocytoplasmic O-Man glycosylation found on proteins and enzymes catalyzing involved in oxidative phosphorylation, including, but not limited to SDHA, SDHB, SDHC, SDHD, ISP, COB, CYT1, COR1, QCR2, QCR6, QCR7, QCR8, QCR9, QCR10, COX1-4, COX5A, COX5B, COX6A, COX6B, COX7A, COX7C, COX10, COX11, COX15, ATP1-9 and ATP14-21 to modulate energy homeostasis, proton gradients and/or adenosine triphosphate (ATP) levels.

In yet another embodiment the present invention relates to modulation of nucleocytoplasmic O-Man glycosylation found on plasma membrane and/or vacuolar ATPase protein complexes, including, but not limited to, PMA1, VMA1-8, VMA10, VMA13 and STV1 to enhance, maintain or inhibit cellular processes by modulating intracellular and/or vacuolar pH levels.

In yet another embodiment the present invention relates to modulation of nucleocytoplasmic O-Man glycosylation found on enzymes catalyzing metabolism of glycogen and trehalose, including, but not limited to, GSY1, GSY2 and GPH1, to modulate levels of storage carbohydrates and/or downstream carbon metabolites.

In yet another embodiment the present invention relates to modulation of nucleocytoplasmic O-Man glycosylation found on enzymes catalyzing nitrogen metabolism, including, but not limited to, GLT1, GDH1-3 and GLN1 to modulate nitrogen metabolism and/or downstream nitrogen metabolites.

In yet another embodiment the present invention relates to modulation of nucleocytoplasmic O-Man glycosylation found on enzymes catalyzing lipid metabolism to modulate lipid metabolism and/or downstream lipid metabolites. These enzymes are known to one skilled in the art, e.g., [_INSERT ENZYMES RELATED TO LIPID METABOLISM_].

In yet another embodiment the present invention relates to modulation of nucleocytoplasmic O-Man glycosylation found on enzymes catalyzing biosynthesis of glycosyl-phosphatidylinositol (GPI) membrane anchors, including, but not limited to, GPI1-3, GPI15, GPI12, YJR013w, GPI10, SMP3, GPI13, GPI17, GPI11, GAA1, GPI8, GPI16, GPI17 and YLR459w to modulate GPI-anchor and/or GPI-anchored protein synthesis.

In yet another embodiment the present invention relates to modulation of nucleocytoplasmic O-Man glycosylation found on carbohydrate sensors including, but not limited to, SNF3 and/or RGT2, to modulate downstream cellular signaling.

In yet another embodiment the present invention relates to modulation of nucleocytoplasmic O-Man glycosylation found on enzymes that indirectly or directly control levels of secondary messenger molecule cyclic adenosine monophosphate (cAMP) including, but not limited to, RAS1, RAS2, CDC25, IRA1, IRA2 and CYR1, to modulate downstream cellular signaling.

### BRIEF DESCRIPTION OF THE DRAWINGS AND TABLES

FIG. 1 illustrates the identification of nucleocytoplasmic protein O-mannosylation in yeast. FIG. 1A: Graphic depiction of the glycoproteomic approach for identification of yeast O-mannosylation. Wild-type (WT) or kre2Δktr1Δktr3Δ mutant (Mut) yeast total cell lysates treated with trypsin and PNGase F are enriched by Concanavalin A (ConA) lectin weak affinity chromatography (LWAC) and glycopeptides identified by mass spectrometry (MS). FIG. 1B: Pie chart showing the cellular localization of identified proteins with O-Man glycosites from total cell lysates of WT- and Mut S. cerevisiae and a cytoplasmic preparation (S3) from WT S. cerevisiae. The composite results (Total) illustrate total non-redundant number of O-Man glycoproteins identified. FIG. 1C: Gas chromatography-mass spectrometry (GC-MS) analysis of mannitol and sorbitol standards (gray trace). GC-MS profile of yeast reducing-end sugars released by reductive β-elimination (black trace). FIG. 1D: Analysis of O-Man anomericity using Jack bean α-mannosidase. Bar chart shows total (nuclear, cytoplasmic and mitochondrial) number of identified O-Man glycoproteins for WT and Mut S. cerevisiae strains before (−) and after (+) α-mannosidase treatment.

FIG. 2 illustrates cross-talk between O-Man glycosylation and phosphorylation with analogy to O-GlcNAcylation to higher eukaryotes. FIG. 2A: Sequence alignment (grayscale proportional to conservation) of human and yeast glycogen phosphorylase with expansion of the N-terminal domain showing the co-occupancy at the regulatory Thr31 residue. FIG. 2B: Alignment of human and yeast histone H2B proteins demonstrating C-terminal O-GlcNAc and O-Man modifications, respectively. Human H2B lysine 120 indicated in yellow. FIG. 2C: Alignment of mouse and yeast plasma membrane ATPase proteins. Left expansion shows part of the ATP binding motif (yellow) with overlapping O-GlcNAc/O-Man occupancy at Thr558. Right expansion shows the C-terminal regulatory domain modified by O-Man and phosphorylations.

FIG. 3 Illustrates mass spectrograms identifying the epimeric configuration of hexose monosaccharides released from yeast fraction S3. FIG. 3A: Gas chromatography-mass spectrometry of sorbitol, mannitol and mannose standards (upper panel) together with hexoses from yeast S3 fraction released by reductive β-elimination (lower panel). Automatic area (AA) integration was performed to estimate the relative amounts of mannose (84%) and mannitol (16%) from the yeast S3 sample. All samples have been spiked with myo-inositol (internal standard), depolymerized by acid treatment and TMS-derivatized prior to injection. Retention times are normalized relative to the myo-inositol standards. FIG. 3B: Mass spectra from peaks at ˜30 min in FIG. 3A showing the TMS-hexitol fragmentation pattern for sorbitol, mannitol and yeast S3 (mannitol).

FIG. 4 Illustrates graphically the distance of glycosites of different types of O-glycosylation to the closest phosphorylation site in proteins. FIG. 4: Unambiguously assigned site data for mammalian O-GlcNAc (802 proteins, 2601 sites) and S. cerevisiae O-Man data (92 cytosolic proteins, 202 sites and 151 extracellular proteins, 855 sites), as well as phosphorylation data from (18-21) were used to find the distance to the closest phosphorylation site within the same compartment (determined using transmembrane region prediction from TMHMM) up to a distance of 200 amino acids. A higher proportion of cytosolic proteins bearing O-Man have nearby phosphorylation sites than the set of extracellular O-Man proteins. The cytosolic O-Man have a higher proportion of sites closer to phosphorylation sites, similar to O-GlcNAc, suggesting a similar relationship between O-Man glycosylation and phosphorylation as found for O-GlcNAc.

FIG. 5 Illustrates WebLogo plots comparing chemical properties of neighbouring amino acids for FIG. 5A: mammalian O-GlcNAc (2371 sites), FIG. 5B: yeast nucleocytoplasmic O-Man (202 sites), and FIG. 5C: yeast extracellular O-Man sites (855 sites). FIG. 5D: Key for FIG. 5A-5C: Amino acids are encoded such that “S” encodes for small and nonpolar residues (S,T,G,A); “A” encodes for acidic residues (D,E); “B” encodes for basic residues (K,R,H); “P” encodes for polar residues (N,Q,Y) and “H” encodes for hydrophobic residues (C,F,I,L,M,P,V,W). The O-GlcNAc neighbourhood is substantially similar to the previously published logo plot (24).

FIG. 6 Illustrates histograms of quantitative analysis of yeast O-Man glycosites after nutrient deprivation. FIG. 6A: Short burst of nutrient deprivation does not significantly affect the proteome as demonstrated by fold-change (Log 10) of dimethyl labelled peptide ratios (medium/light) of stimulated (glucose deprived; medium label) and non-stimulated (control; light label). FIG. 6B: Distinct changes of the nucleocytoplasmic O-Man glycosylations following nutrient deprivation. The significantly downregulated (>100 fold change) O-Man glycopeptides were all derived from proteins localized to nucleocytoplasmic compartments. ER/Golgi derived O-Man glycosylations were not identified among >100 fold downregulated O-Man glycosylations, thus showing that they are static with no significant changes in this experimental setup.

FIG. 7 Illustrates HPLC chromatographs demonstrating incorporation of ¹⁴C-mannose into a peptide substrate by a soluble alpha-mannosyltransferase activity. FIG. 7A): HPLC chromatograph of the LKNVPTPSPSPKPQH acceptor substrate peptide alone; FIG. 7B: HPLC chromatograph of the enzyme reaction containing a yeast cell lysate and the LKNVPTPSPSPKPQH peptide as described in Example 6; FIG. 7C: Counts (¹⁴C by scintillation counter) of fractions from HPLC separation of the reaction mixture in B using GDP-¹⁴C-Man as donor substrate. All reactions were passed through Dowex weak ion exchange column before HPLC analysis to remove unused GDP-Man.

TABLE 1 lists the identified nucleocytoplasmic O-Man glycoproteins from S. cerevisiae and S. pombe. The table includes protein identifiers (Uniprot ID and Systematic ID), gene names (Gene) together with ambiguously and unambiguously identified O-Man glycosylation sites numbered according to protein sequence (Sites).

### DETAILED DESCRIPTION OF THE INVENTION

The practice of the present invention will employ, unless indicated specifically to the contrary, conventional methods of molecular biology, recombinant DNA techniques, protein expression, and protein/peptide/carbohydrate chemistry within the skill of the art, many of which are described below for the purpose of illustration. Such techniques are explained fully in the literature. See, e.g., Sambrook, et al., Molecular Cloning: A Laboratory Manual (3rd Edition, 2000); DNA Cloning: A Practical Approach, vol. I & II (D. Glover, ed.); Oligonucleotide Synthesis (N. Gait, ed., 1984); Oligonucleotide Synthesis: Methods and Applications (P. Herdewijn, ed., 2004); Nucleic Acid Hybridization (B. Hames & S. Higgins, eds., 1985); Nucleic Acid Hybridization: Modern Applications (Buzdin and Lukyanov, eds., 2009); Transcription and Translation (B. Hames & S. Higgins, eds., 1984); Animal Cell Culture (R. Freshney, ed., 1986); Freshney, R.I. (2005) Culture of Animal Cells, a Manual of Basic Technique, 5th Ed. Hoboken N.J., John Wiley & Sons; B. Perbal, A Practical Guide to Molecular Cloning (3rd Edition 2010); Farrell, R., RNA Methodologies: A Laboratory Guide for Isolation and Characterization (3rd Edition 2005). Poly(ethylene glycol), Chemistry and Biological Applications, ACS, Washington, 1997; Veronese, F., and J. M. Harris, Eds., Peptide and protein PEGylation, Advanced Drug Delivery Reviews, 54(4) 453-609 (2002); Zalipsky, S., et al., “Use of functionalized Poly(Ethylene Glycols) for modification of polypeptides” in Polyethylene Glycol Chemistry: Biotechnical and Biomedical Applications. The publications discussed above are provided solely for their disclosure before the filing date of the present application. Nothing herein is to be construed as an admission that the invention is not entitled to antedate such disclosure by virtue of prior invention.

### Definitions

Unless defined otherwise, all technical and scientific terms used herein have the same meaning as commonly understood by one of ordinary skill in the art to which this invention belongs. As used in the specification and appended claims, unless specified to the contrary, the following terms have the meaning indicated. With regard to this specification, any time a definition of a term as defined herein, differs from a definition given for that same term in an incorporated reference, the definition explicitly defined herein is the correct definition of the term.

The words “a” and “an” denote one or more, unless specifically noted.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. In any embodiment discussed in the context of a numerical value used in conjunction with the term “about,” it is specifically contemplated that the term about can be omitted.

As used herein, the term “amino acid” is intended to mean both naturally-occurring and non-naturally-occurring amino acids, as well as amino acid analogs and chemical mimetics. Naturally-occurring amino acids include the 20 (L)-amino acids utilized during protein biosynthesis as well as other amino acids formed during post-translational modification such as, for example, 4-hydroxyproline, hydroxylysine, desmosine, isodesmosine, homocysteine, citrulline and ornithine. Non-naturally-occurring amino acids include, for example, (D)-amino acids, norleucine, norvaline, p-fluorophenylalanine, ethionine and the like, which are known to a person skilled in the art. Amino acid analogs include modified forms of naturally and non-naturally-occurring amino acids. Such modifications can include, for example, substitution or replacement of chemical groups and moieties on the amino acid or by derivatization of the amino acid. Amino acid mimetics include, for example, organic structures that exhibit functionally similar properties such as charge and charge spacing characteristic of the reference amino acid. For example, an organic structure which mimics Arginine (Arg or R) would have a positive charge moiety located in similar three-dimensional molecular space and having the same degree of mobility as the ε-amino group of the side chain of the naturally-occurring Arg amino acid. Mimetics also include constrained structures so as to maintain optimal spacing and charge interactions of the amino acid or of the amino acid functional groups. Those skilled in the art know or can determine what structures constitute functionally equivalent amino acid analogs and amino acid mimetics.

Reference to the term “e.g.” is intended to mean “e.g., but not limited to” and thus it should be understood that whatever follows is merely an example of a particular embodiment, but should in no way be construed as being a limiting example. Unless otherwise indicated, use of “e.g.” is intended to explicitly indicate that other embodiments have been contemplated and are encompassed by the present invention.

Reference throughout this specification to “embodiment” or “one embodiment” or “an embodiment” or “some embodiments” or “certain embodiments” means that a particular feature, structure or characteristic described in connection with the embodiment is included in at least one embodiment of the present invention. Thus, the appearances of the phrases “in one embodiment” or “in an embodiment” or “in certain embodiments” in various places throughout this specification are not necessarily all referring to the same embodiment. Furthermore, the particular features, structures, or characteristics may be combined in any suitable manner in one or more embodiments.

By a polypeptide having an amino acid sequence at least, for example, 95% “identical” to a query amino acid sequence (e.g., a amino acid sequence that can be compared to published sequences using the Basic Local Alignment Search Tool or BLAST algorithm; Johnson M, Zaretskaya I, Raytselis Y, Merezhuk Y, McGinnis S, Madden TL (2008) NCBI BLAST: a better web interface. Nucleic Acids Res. 36:W5-W9, incorporated herein) of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

An “increased” or “enhanced” amount is typically a “statistically significant” amount, and may include an increase that is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 2.1, 2.2, 2.3, 2.4, etc.) an amount or level described herein. Similarly, a “decreased” or “reduced” or “lesser” amount is typically a “statistically significant” amount, and may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein.

As used herein, “native sequence” or “native polynucleotide” or “native polypeptide” refers polynucleotide or polypeptide that is found in nature. In some embodiments, “native sequence” or “native polypeptide” refers to any primary accepted reference sequence according to the UniProt Knowledgebase database (UniProt Consortium. Update on activities at the Universal Protein Resource (UniProt) in 2013. Nucleic Acids Res. 2013 41:D43-7, incorporated herein) or the UniGene database (Pontius J U, Wagner L, Schuler G D. UniGene: a unified view of the transcriptome. In: The NCBI Handbook. Bethesda (Md.): National Center for Biotechnology Information; 2003, incorporated herein). The UniProt database can be found at the world wide web address uniprot.org, the contents of which are incorporated herein. The UniGene database can be found at the world wide web address ncbi.nlm.nih.gov/unigene, the contents of which are incorporated herein.

“Nucleocytoplasmic O-mannosylation” refers to the process of attaching an alpha-linked mannose to a polypeptide. In various embodiments, nucleocytoplasmic O-mannosylation refers to the enzymatically-catalysed process of adding an alpha-linked mannose to a polypeptide.

“Nucleocytoplasmic alpha-mannosyltransferase enzyme activity” refers to the enzyme activity that catalyzes nucleocytoplasmic O-mannosylation, e.g., the attachment of an alpha-linked mannose to a polypeptide.

“Optional” or “optionally” means that the subsequently described event, or circumstances, may or may not occur and that the description includes instances where said event or circumstance occurs and instances in which it does not.

The terms “polynucleotide”, “nucleotide”, “nucleotide sequence”, and “nucleic acid” are used interchangeably. They refer to a polymeric form of nucleotides of any length, either deoxyribonucleotides or ribonucleotides, or analogs thereof. Polynucleotides may have any three dimensional structure, and may perform any function known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may include non-nucleotide components. A polynucleotide may be further modified after polymerization, such as by conjugation with a labeling component.

The terms “polypeptide” or “peptide” or “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example by disulfide bond formation, glycosylation, lipidation, acetylation, phosphorylation, hydroxylation, or any other manipulation, such as conjugation with a labelling component.

### Overview

All eukaryotes except nematodes and plants have a well-characterized O-Man glycosylation machinery for proteins trafficking the secretory pathway, and the enzymes involved in this process are multi-transmembrane spanning Dol-P-Man:protein O-mannosyltransferases (PMTs) utilizing the membrane associated dolichol phosphate β-D-Man (Dol-P-Man) donor substrate and transferring a single Man residue to selected Ser and Thr residues of proteins. These enzymes are located in the ER with their catalytic domains oriented into the lumen (Lommel and Strahl 2009, Loibl and Strahl 2013). Higher eukaryotic cells have two PMT isoenzymes (POMT1 and POMT2) and the initial O-Man glycans are elongated, branched and capped by sialic acids by a series of glycosyltransferases. Deficiencies in many of the enzymes involved in protein O-mannosylation in humans underlie a group of congenital muscular dystrophies (Endo 2015). Yeast in contrast have a family of at least six PMTs, and the initial Man monosaccharide is extended only by additional mannose residues through the actions of Golgi resident mannosyltransferases that use guanosine diphosphate α-D-Man (GDP-Man) as donor substrate (Loibl and Strahl 2013). The O-Man glycosylation and the action of multiple PMTs are essential for yeast (Gentzsch and Tanner 1996), and O-Man plays major roles in maintaining yeast cell wall integrity (Arroyo, Hutzler et al. 2011).

Current knowledge of the proteins undergoing O-Man glycosylation in yeast and the specific sites of glycosylation is limited (Lommel and Strahl 2009, Loibl and Strahl 2013), but recently we developed a glycoproteomics strategy to probe the O-Man glycoproteome of human cells using genetic engineering to simplify the O-glycan structures, the so-called “SimpleCell” strategy, in combination with Concanavalin A (ConA) lectin chromatography for enrichment of glycopeptides and mass spectrometric sequencing (Vester-Christensen, Halim et al. 2013). This resulted in identification of a large number of O-Man glycoproteins and O-Man glycosites demonstrating for example that cadherins and protocadherins are major carriers of O-Man glycans. In the present invention we modified this strategy to probe the yeast O-Man glycoproteome from total cell lysates. In striking contrast to our studies with human cell lines, we in addition to proteins entering the secretory pathway, also identified a large number of O-Man proteins annotated as classical nuclear, cytosolic or mitochondrial proteins that are not expected to be exposed to the known O-Man glycosylation machinery in the secretory pathway. The nucleocytoplasmic O-Man glycosites were located on proteins and in positions resembling that of the O-GlcNAcylation process in higher eukaryotes. We characterized the O-Man glycoproteome and found evidence to support that the nucleocytoplasmic O-Man modifications in yeast represents the missing equivalent to the O-GlcNAcylation process of higher eukaryotes.

The present invention relates, generally, to compositions and methods related to nucleocytoplasmic O-mannosylation. In particular embodiments, the present invention relates, generally, to compositions and methods related to nucleocytoplasmic O-mannosylation in yeast.

In some embodiments, the present invention provides nucleocytoplasmic O-Man glycoproteins and O-Man glycosites as targets for modulation of the biological functions of O-mannosylation in yeast.

In some embodiments this entails mutagenesis of an individual or a plurality of O-Man glycosites. Methods of mutagenesis are known in the art. In some embodiments, such mutagenesis results in modulation of yeast growth, nutrient sensing, fermentation, and/or bioproduction.

In some embodiments the present invention provides a modified polypeptide comprising a mutation in one or more O-Man glycosites. In some embodiments, suitable mutations include deletions, additions, or substitutions of any one or more native amino acid sequence in a native O-mannosylated nucleocytoplasmic polypeptide, wherein the mutation results in modulation of nucleocytoplasmic O-mannosylation of the polypeptide. Methods of making deletions, additions, and substitutions in a polypeptide sequence, e.g., via genetic engineering techniques such as site directed mutagenesis are known in the art. In some embodiments the present invention provides a modified polypeptide listed on Table 1, wherein the polypeptide is modified to comprise a mutation in one or more of the O-Man glycosites identified in Table 1.

As used herein, reference to “the modulation of nucleocytoplasmic O-mannosylation” or “modulated nucleocytoplasmic O mannosylation” refers to increasing or decreasing the O-Man occupancy of one or more O-Man glycosite on one or more polypeptide expressed in the nucleocytoplasmic compartment.

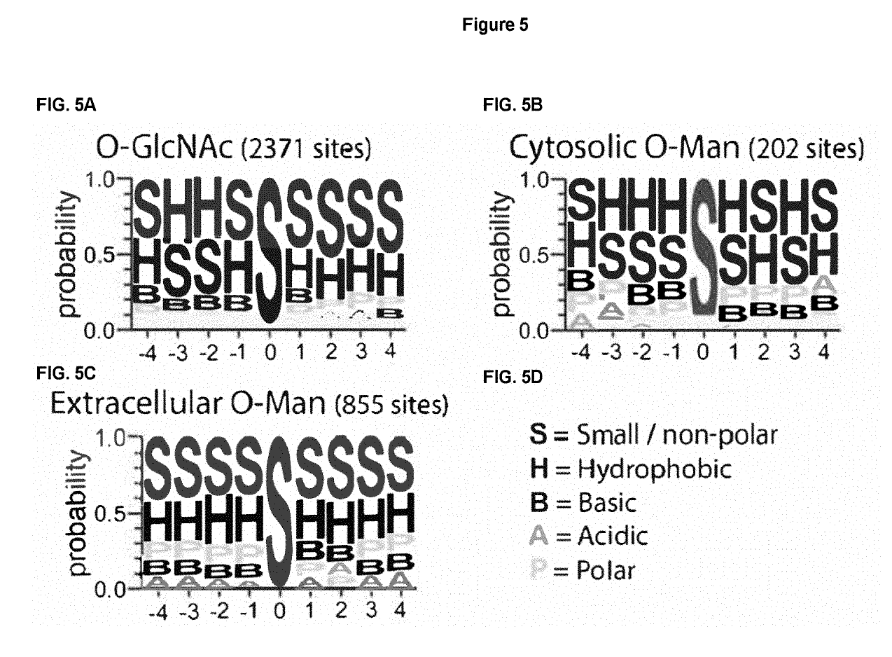

In certain embodiments, the mutation comprises a deletion or substitution of an O-Man glycosite, wherein the deletion or substitution ablates O-mannosylation of the glycosite. In certain embodiments, the mutation comprises a deletion or substitution of an amino acid residue that controls O-mannosylation of the O-Man glycosite, such that the deletion or substitution ablates or reduces O-mannosylation of the glycosite. An amino acid that “controls O-mannosylation” is an amino acid in an O-mannosylated nucleocytoplasmic protein, which is not itself an O-Man glycosite, but the presence or absence of which amino acid dictates whether, or to what extent, an O-Man glycosite is mannosylated. The skilled artisan will fully appreciate that glycosylation, like phosphorylation, is to some extent influenced by the peptide backbone sequence of the target polypeptide. The presence or absence of preferred or non-preferred sequences near or adjacent to a potential glycosite influences the efficiency at which a glycosyltransferase can glycosylate the glycosite. For example, as shown in FIG. 5, a higher proportion of flanking hydrophobic residues was observed for nucleocytoplasmic O-Man glycosites compared to O-Man glycosites found on extracellular proteins.

In certain embodiments, the mutation comprises an addition or substitution of an O-Man glycosite or an amino acid that controls O-mannosylation, wherein the addition or substitution increases or induces O-mannosylation of a glycosite present in a polypeptide expressed in the nucleocytoplasmic compartment or wherein the addition or substitution introduces a non-native amino acid into a polypeptide expressed in the nucleocytoplasmic compartment such that the non-native amino acid is O-mannosylated.

In another embodiment the invention relates to methods to modulate the nucleocytoplasmic O-Man glycosylation process for example by nutrient deprivation.

In some embodiments, the invention relates to methods of modulating the nucleocytoplasmic O-Man glycosylation process for example by altering access to the donor substrate GDP-Man.

In some embodiments, the invention relates to methods of modulating the nucleocytoplasmic O-Man glycosylation process for example by altering access to glucose.

In some embodiments, nutrient deprivation, e.g., restricted access to glucose or GDP-Man, results in an increase in the O-mannosylation of a polypeptide.

In other embodiments, nutrient deprivation e.g., restricted access to glucose or GDP-Man, results in an increase in the O-mannosylation of a polypeptide.

In another embodiment the inventions relates to methods to modulate the O-Man glycosylation process for example by inhibitors or activators of the O-Man glycosyltransferase activity catalyzing the transfer of Man to polypeptides in the nuclear and cytoplasmic space found in yeast.

### EXAMPLES

The purpose of the following examples are given as an illustration of various embodiments of the invention and are thus not meant to limit the present invention in any way. Along with the present examples the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### Determining the O-Man Glycoproteome of S. cerevisiae and S. pombe

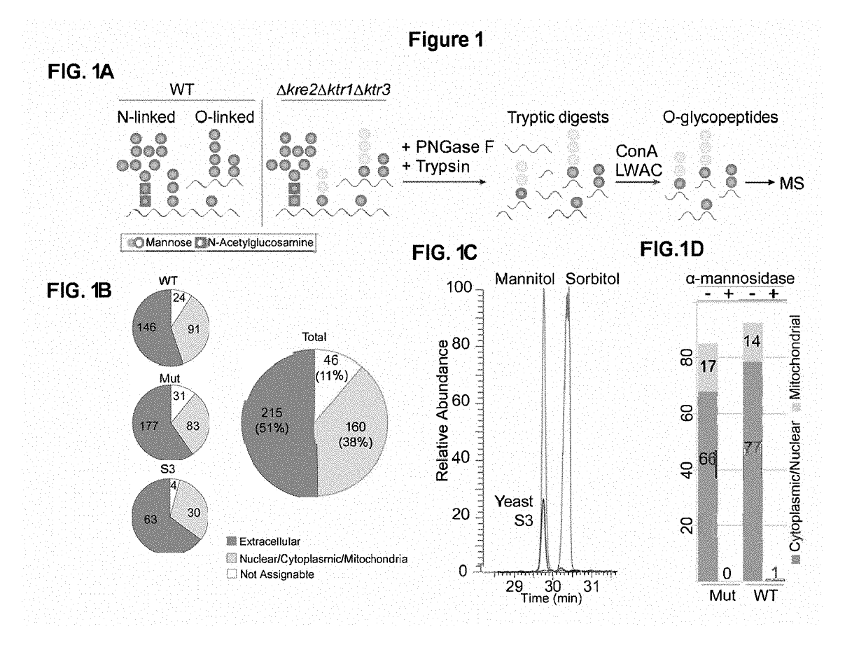

Since yeast is known to produce heterogeneous elongated poly-mannose structures, we initially used the kre2zΔktr1Δktr3Δ mutant strain (Mut), lacking the α1,2-mannosyltransferase (kre2) involved in the second biosynthetic step of O-Man glycans and additional gene family members (ktr1 and ktr3), to enable analysis of the O-Man glycoproteome with more simplified O-Man glycan structures (FIG. 1A) resembling the SimpleCell strategy (Vester-Christensen, Halim et al. 2013).

We used total cell lysates obtained by vortexing with glass beads in Rapigest detergent for trypsin digestion. N-linked glycans were removed by PNGase F digestion and the digests were subjected to Concanavalin A (ConA) lectin weak affinity chromatography (LWAC) for enrichment of O-Man glycopeptides. Enriched O-Man glycopeptides were further fractionation by isoelectric focusing (IEF) and analyzed by nLC-MS/MS (FIG. 1A). Through this approach using the kre2Δktr1Δktr3Δ mutant strain, we identified a considerable number of O-glycoproteins and O-glycosites with one or more hexoses attached utilizing both higher-energy collision dissociation (HCD) and electron transfer dissociation (ETD) fragmentation modes. A summary of the identified glycoproteins from S. cerevisiae is presented in FIG. 1B. In total we mapped 291 unique O-Man glycoproteins and approximately 1,000 O-Man glycosites in Mut total cell lysates.

### Methods

### Yeast Strains and Culture Conditions.

The following Saccharomyces cerevisiae strains were used: wild-type (BY4741, MATa his3Δ1 leu2Δ0 met15Δ0 ura3Δ0), the KTR/MNT triple mutant (BY4741 except kre2Δ::His3-GFP, ktr1Δ::SAT, ktr3Δ::KanMX4, a gift of H. Bussey) and BY5457 loxp::pep4Δ::loxp (wild type BY5457 (MATα ura3-52::p1785 leu2-3,112 his4-519 trp1 pho3-1 pho5-1 canr) with the additional loxp::pep4Δ::loxp modification, a gift from Rosa Laura Lopez Marques). WT cells (BY4741) were grown in rich medium (YPD) containing 2% glucose, 2% peptone, and 1% yeast extract under aerobic conditions at 30° C. with constant shaking at 170 rpm in a rotary shaker incubator (I Series 26, New Brunswick Scientific Co., Inc., Edison, N.J., USA). Cell growth was determined by measuring the optical density at a wavelength of 600 nm (OD600). S. cerevisiae cultures were grown to mid-log phase (OD600 0.8 to 1.1) and harvested by centrifugation at 3,000×g for 5 min. Growth conditions and harvesting of BY5457 cells is described below. The Schizosaccharomyces pombe WT (no-marker h−) strain, a derivative of the WT heterothallic strains 972h- and 975h+, was used. The fission yeast cells were propagated at 30° C. in YES media (5 g/L yeast extract, 30 g/L glucose, 225 mg/L adenine, 225 mg/L leucine, 225 mg/L uracil) and harvested in the late exponential phase by centrifugation (3000 g, 2 min) and lysed using glass beads as described below.

### LWAC Isolation of O-Man Glycopeptides.

Cell extracts were prepared from a total of 100 OD600 units of packed yeast cells with acid washed 5 mm glass beads in ice-cold 0.1% Rapigest (Waters Corp., Milford, Mass., USA) in 50 mM ammonium bicarbonate by vortexing in reciprocal shaker (Hybaid RiboLyser) for 4×25 s with 1 min intervals at 4° C. The bottom of the tube was punctured, and the lysate was collected. Unbroken cells and larger cell debris were removed by a low-speed centrifugation step at 1,500×g for 5 min at 4° C. The cleared lysates were heated at 80° C., 10 min, followed by reduction in 5 mM dithiothreitol at 60° C., 30 min and alkylation in 10 mM iodoacetamide at room temperature (RT), in darkness and 30 min. Samples were digested with 25 μg trypsin (Roche) over night (ON), heat-inactivated by incubation at 95° C., 20 min and treated with 8 U PNGase F ON at 37° C. An additional 4 U PNGase F was added and incubated at 37° C. for 4 h. The digests were acidified with 12 μl TFA, incubated at 37° C., 20 min, cleared by centrifugation at 10,000×g, 10 min and purified by Sep-Pak C18 (Waters) columns. The LWAC protocol for isolation of O-Man glycopeptides was as previously described (Vester-Christensen, Halim et al. 2013). Sep-pak purified peptides were concentrated by evaporation and the reduced solution was diluted with an equal volume of 2× Con A buffer A (40 mM Tris HCl, pH 7.4, 300 mM NaCl, 2 mM CaCl2/MgCl2/MnCl2/ZnCl2, 1 M urea) before loading in a 2.8 m long Con A lectin agarose column. The column was washed with 10 column volumes (CV) ConA buffer A at 100 μl/min before elution with 5 CV's ConA buffer B (20 mM Tris HCl, pH 7.4, 150 mM NaCl, 1 mM CaCl2/MgCl2/MnCl2/ZnCl2, 0.5M methyl-α-D-glucopyranoside/methyl-α-D-mannopyranoside) at 50 μl/min. Fractions containing glycopeptides were purified by in-house packed Stage tips (Empore disk-C18, 3M) and further fractionated by isoelectric focusing (IEF) as previously described (Vakhrushev, Steentoft et al. 2013). For each IEF fraction, 50% was analysed by nLC/MS/MS as described below. The remaining 50% of each IEF fraction was digested at 37° C. ON with 30 U/ml Jack bean α-mannosidase (Prozyme) in 100 mM sodium acetate, 2 mM Zn2+, pH 5 prior to analysis by nLC/MS/MS.

nLC/MS/MS and Data Analysis.

Mass spectrometric analyses were performed essentially as previously described (Vester-Christensen, Halim et al. 2013). Samples were analyzed on a set up composed of an EASY-nLC 1000 (Thermo Fisher Scientific) interfaced via a nanoSpray Flex ion source to an LTQ-Orbitrap Velos Pro hybrid spectrometer (Thermo Fisher Scientific). The EASY-nLC 1000 was equipped with a polar end-capped C18-silica column; 21 cm length, 75 μm inner diameter and 1.9-μm particle size. A data-dependent mass spectral acquisition routine, HCD and subsequent ETD scan, was used for all runs. Briefly, a precursor MS1 scan (m/z 355-1,700) of intact peptides was acquired in the Orbitrap at a resolution setting of 30,000, followed by Orbitrap HCD-MS2 and ETD-MS2 of the five most abundant multiply charged precursors in the MS1 spectrum; a minimum MS1 signal threshold of 50,000 ions was used for triggering data-dependent fragmentation events; MS2 spectra were acquired at a resolution of 15,000. Data processing was carried out using Proteome Discoverer 1.4 software (Thermo Fisher Scientific) as previously described (Vester-Christensen, Halim et al. 2013) with minor modifications as outlined below. Raw data files (.raw) were processed using the Sequest HT node and searched against the canonical S. cerevisiae proteome (7225 entries) downloaded from the Uniprot database (October, 2013) or the canonical S. pombe proteome (5092 entries; September 2015). Spectral assignments at the medium confidence level (p>0.01) and below were resubmitted to a second Sequest HT node using semi-specific tryptic cleavage. Final results were filtered for high-confidence (p<0.01) identifications only. Spectra matched to peptides with nucleocytoplasmic O-Man modifications were inspected manually to verify the accuracy of the assignments. The mass spectrometry proteomics data have been deposited to the ProteomeXchange Consortium (Vizcaino, Deutsch et al. 2014) via the PRIDE partner repository with the dataset identifier PXD002924. Signal peptides were retrieved manually from Uniprot or through the SignalP tool (server version 4.1); transmembrane domains were predicted with TMHMM (server version 2.0); both tools available at http://www.cbs.dtu.dk/services/.

Cytoplasmic proteins (S3 fraction) were prepared as follows: Cells (BY5457 loxp::pep4Δ::loxp) were grown as above in YPAD medium (YPD supplemented with 0.01% Adenine hemisulphate w/v). Cells were pelleted by centrifugation at 800×g, 15 min, 4° C., and suspended in a 1:5 ratio in 50 ml lysis buffer (50 mM Tris-HCl pH 7.5, 150 mM NaCl) supplemented with 1 tablet complete protease inhibitor (Roche) and lysed by French press at 4° C., 40 Kpsi (S1 fraction). The S1 fraction was cleared by centrifugation at 5000×g, 4° C., 15 min. The supernatant (S2) was subjected to ultracentrifugation at 45.000×g at 4° C., 30 min, followed by 100.000×g at 4° C., 3 h, resulting in the cytoplasmic S3 (supernatant) fraction. The S3 fraction was trypsin digested, ConA enriched and purified by Stage tips as described above. For each LWAC elution fraction, 5% of total sample was injected and analyzed by HCD fragmentation only.

### MALDI and GC-MS Analysis.

The S3 fraction (13 ml) was dialyzed against 6 L 50 mM NH4HCO3, pH 7.8, using 8,000 Da MWCO membranes and concentrated by evaporation. Following reduction and alkylation (described above), the cytosolic proteins were trypsin digested ON and purified by Sep-Pak C18 (Waters) columns. The tryptic peptides were dried, resolubilized in 100 mM NaOH, 1 M NaBH4 and incubated ON at 50° C. The β-elimination reaction was terminated by the addition of 8 μL glacial acetic acid and the released O-glycan alditols were separated from proteins by a second Sep-Pak C18 (Waters) purification. Reduced O-glycans were desalted by Dowex AG 50W X8 cation exchange resin (Bio-Rad) followed by repeated (×5) addition of 500 μL 1% acetic acid in methanol and evaporation over a stream of N2 gas. For MALDI analysis, released oligosaccharides were permethylated essentially as previously described (Ciucanu and Costello 2003). Briefly, released oligosaccharides were dried in glass vials to which 18 mg NaOH powder, 150 μl dimethyl sulfoxide (DMSO) with 0.1% H₂O (v/v) and 30 μl methyl iodide was added; the mixture was incubated at RT, 1 h and the reaction was terminated by addition of 150 μl ice cold H₂O followed by 200 μl chloroform. The organic phase was washed five times with 1 ml H₂O and finally dried with a stream of N2 gas. Permethylated oligosaccharides were reconstituted in 30 μl 50% methanol in H₂O (v/v) and 1 μl was co-crystallized with an equal amount of matrix (10 mg/ml 2,5-dihydroxybenzoic acid (DHB) and 2.5 mM sodium acetate in 70% acetonitrile in H2O (v/v)). MALDI TOF (Autoflex Speed, Bruker Daltonics, Bremen, Germany) was operated in the reflector mode using positive polarity and 2000 laser shots/spot. For GC-MS, reduced O-glycans were spiked with 1 μg myo-inositol (internal standard) and depolymerized by incubation in 0.5N methanolic HCL (Supelco), 80° C., 16 h. Monosaccharides were per-O-trimethylsilylated with Tri-Sil reagent (Thermo Scientific) at 80° C., 30 min. GC-MS analysis was performed using a TRACE™ GC Ultra gas chromatograph coupled to a PolarisQ ion trap mass spectrometer. Samples were injected (splitless mode) at 40° C. (1 min), oven temperature was ramped to 150° C. (25° C./min) followed by an increase to 200° C. (1° C./min) before a final ramp to 260° C. (10° C./min) where it was held for 5 min. Monosaccharides were identified by comparison of retention times and mass spectra to hexose and hexitol standards. All retention times were relative to the myo-inositol internal standard.

### Example 2

### Identification of the Nucleocytoplasmic O-Man Glycoproteome of Yeast

Analysis of the identified proteins using the methods described in Example 1 revealed that a large proportion (n=83, 29%) were known or predicted nuclear and/or cytosolic proteins without recognizable signal peptides. This finding was in striking contrast to our previous analysis of the O-Man glycoproteome of human cells, where all identified glycoproteins were known or predicted to traffic the secretory pathway (Vester-Christensen, Halim et al. 2013). We therefore also inspected all identified glycoproteins with signal peptides for the specific localization of O-glycosites with respect to known or predicted membrane orientation, and found sites predicted to be located in the cytosolic compartment (Table 1). Thus, more than a third of the O-Man glycoproteins identified in Mut S. cerevisiae were classical nuclear, cytosolic or mitochondrial proteins that are not exposed to the known O-Man glycosylation machinery in the secretory pathway (FIG. 1B).

To exclude the possibility that the unexpected finding was related to the kre2Δktr1Δktr3Δ mutant strain, we further explored this by analyzing total cell lysates of wild-type (WT) S. cerevisiae, which resulted in identification of 261 O-Man glycoproteins of which a similar fraction of 91 (35%) glycoproteins were from nuclear, cytoplasmic or mitochondrial compartments. Also, we sought to enrich for cytoplasmic proteins by analyzing a crude cytoplasmic fraction (S3) of fractionated WT yeast, and this resulted in identification of a number of unique nucleocytoplasmic glycoproteins (FIG. 1B, Table 1). In total, we identified 162 unique glycoproteins from Mut and WT S. cerevisiae strains with O-glycosites that were either only found in cytosolic, nuclear or mitochondrial compartments (n=160) or where the sites identified were located in the cytosolic part of transmembrane proteins (n=2).

We also explored the O-Man glycoproteome of the fission yeast S. pombe (WT) using the same experimental approach as above (FIG. 1A, Example 1) with total cell lysates. We identified 178 O-Man glycoproteins of which 87 (49%) are known to have cytosolic, nuclear or mitochondrial localization (Table 1). The identified O-Man proteins classified as nucleocytoplasmic greatly expanded the total nucleocytoplasmic glycoproteome, since there was little overlap among the datasets from S. cerevisiae and S. pombe with only a few identified in both.

We focussed mainly on the deepest nucleocytoplasmic O-Man glycoproteome data obtained from S. cerevisiae (Table 1).

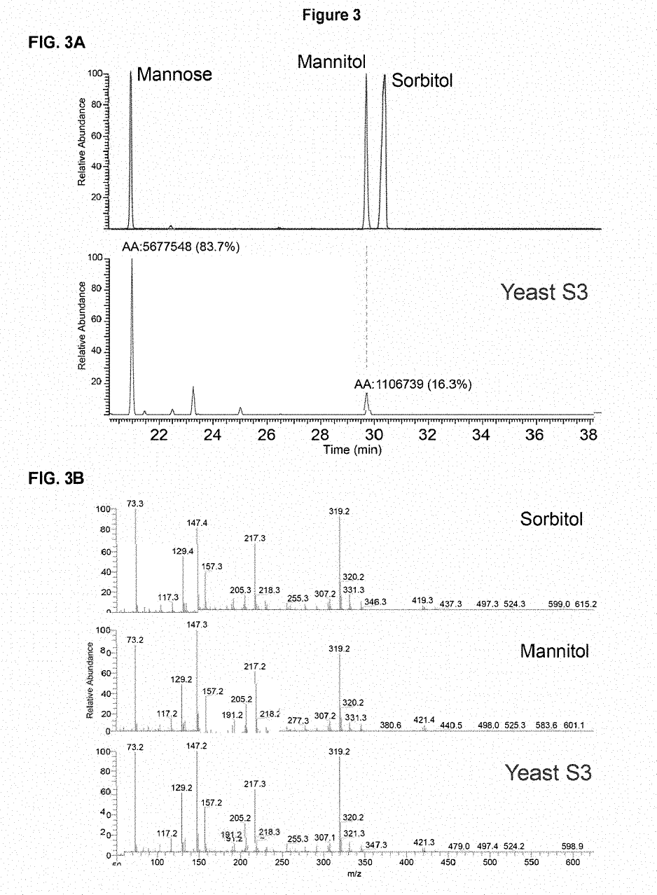

The nucleocytoplasmic O-glycans were shown to be based on Man-α-O-Ser/Thr. Our O-glycoproteomics strategy largely hinges on the α-Mannose specificity of the lectin ConA used for the LWAC as well as the MS identification of the mass increment for hexose residues. Thus, the approach does not reveal the absolute stereochemistry of the identified modifications. We therefore performed a series of experiments to verify the anomeric and epimeric configuration of the identified peptide-linked hexose residue. First, we released O-linked glycans by reductive β-elimination and analyzed the O-glycan profiles by MALDI-TOF mass spectrometry and found Hex₂₋₅ oligosaccharides to be the dominating species. This glycoprofiling does not enable us to selectively quantify the composition of hexose structures on the nucleocytoplasmic glycoproteins, but we identified nucleocytoplasmic proteins with both a single and a disaccharide hexose structure showing that at least some elongation occurs. Mammalian nucleocytoplasmic O-GlcNAc is not elongated by other glycans, but in plants this type of glycosylation is elongated (Heese-Peck, Cole et al. 1995). The glycans released by reductive β-elimination were subsequently subjected to acid hydrolysis and trimethylsilyl derivatization prior to gas chromatography-mass spectrometry (GC-MS) analysis. This approach enables the reducing end hexose to be differentiated from internal or terminal hexose residues through its distinct retention time in GC-MS, and for the β-eliminated yeast O-glycans, the reducing end hexose displayed a similar retention time and fragmentation pattern as the mannitol standard, thus confirming that the peptide-linked hexose is a mannose residue (FIG. 1C and FIG. 3). Having resolved the epimeric configuration of the glycan, we then turned our attention to the anomeric configuration of the mannose-peptide linkage. We performed jack bean α-mannosidase digestion on the ConA enriched and IEF fractionated samples from both WT and kre2Δktr1Δktr3Δ yeast, and found that essentially all mannose residues were hydrolyzed by the α-mannosidase treatment (FIG. 1D). Approximately 25% of the identified O-Man glycopeptides were readily identified as the corresponding peptides without the Man residues after digestion. This demonstrates that the anomericity of the mannose-linkage is in α-configuration.

### Example 3

### Nucleocytoplasmic O-Man Residues are Located in Proximity to Phosphorylation Sites

The 160 identified O-Man nucleocytoplasmic glycoproteins identified in S. cerevisiae included transcription factors, nucleoporins, histones and kinases, which are all classical proteins undergoing O-GlcNAcylation as well as phosphorylation in higher eukaryotes (Table 1). For the identified nucleocytoplasmic yeast glycoproteins, 80% are known to be phosphorylated (Bodenmiller, Campbell et al. 2008, Gnad, Gunawardena et al. 2011, Hornbeck, Kornhauser et al. 2012, Sadowski, Breitkreutz et al. 2013), and as much as 19% (221 in total) of the identified O-Man glycosites were found to be identical to previously identified phosphorylation sites (Table 1), a relatively high number considering that neither the current yeast phosphoproteome nor the O-Man glycoproteome presented here are complete.

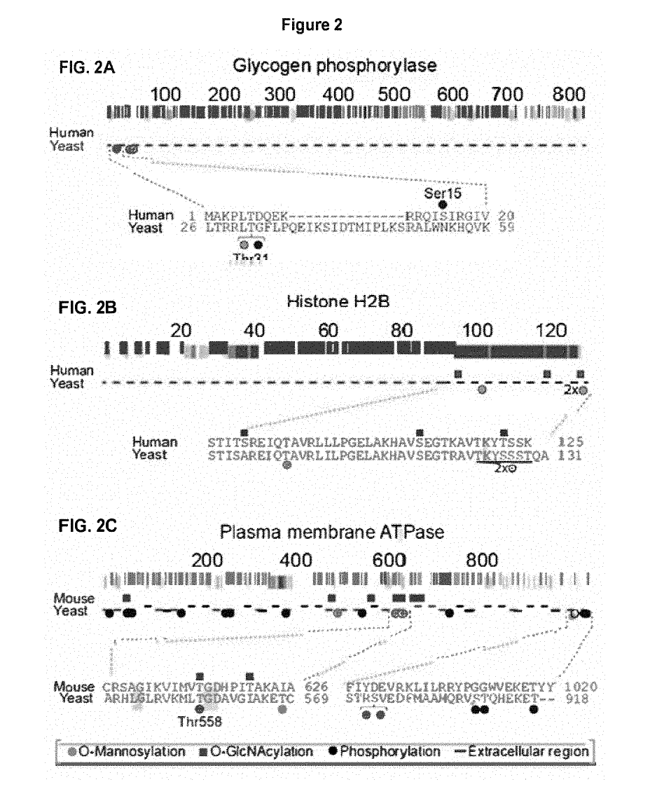

One important example was the glycogen phosphorylase (GP) enzyme, which mobilizes cellular energy by breaking down glycogen into glucose-1-phosphate (G1P). The mechanism of GP activation is conserved among eukaryotes and involves, in addition to allosteric elements, a single phosphorylation at Thr31 in yeast and Ser15 in mammals, both located in the N-terminal regulatory domain (FIG. 2A) (Fosset, Muir et al. 1971, Lin, Hwang et al. 1995). We identified an O-Man glycan on the regulatory Thr31 of yeast GP, demonstrating that nucleocytoplasmic O-Man in yeast may compete directly with phosphorylation for functionally important sites. Clearly O-Man glycosylation of Thr31 in GP will block phosphorylation, and it is expected that this will severely affect activation of the enzyme. Thus, the nucleocytoplasmic O-Man glycosylation may therefore function as an important control switch for utilization of reserve carbohydrates in yeast.

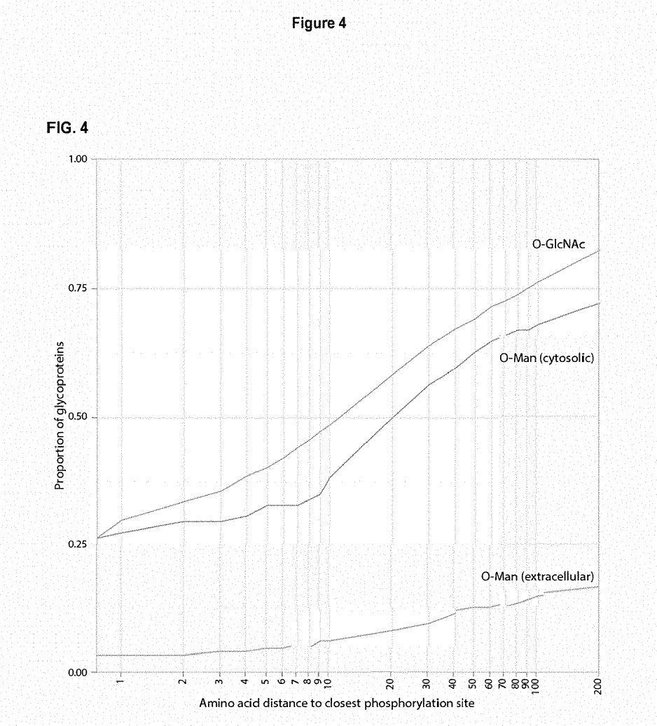

We also compared the proximity of all O-Man glycosites to known phosphorylation sites, which further demonstrated that the O-Man glycosites assigned to nucleocytoplasmic proteins in general tended to have phosphorylation sites closer than the O-Man glycosites assigned to proteins and protein domains exposed to the secretory pathway (FIG. 4). Thus, the nucleocytoplasmic O-Man glycoproteins clearly resemble the mammalian O-GlcNAcylated proteins.

We further analysed the amino acid sequences surrounding the nucleocytoplasmic O-Man glycosites but this approach did not reveal any apparent sequence motifs for glycosylation (FIG. 5) similar to what has been reported for O-GlcNAcylation (Trinidad, Barkan et al. 2012). However, a higher proportion of flanking hydrophobic residues was observed for nucleocytoplasmic O-Man glycosites compared to O-Man glycosites found on extracellular proteins.

Analysis of the subset of proteins for which mammalian orthologs could be reliably established (79/160) revealed that 24% of these have also been reported to be O-GlcNAcylated in rodents or humans (Wang, Torii et al. 2011, Alfaro, Gong et al. 2012, Trinidad, Barkan et al. 2012, Cao, Cao et al. 2013). This analysis is likely biased by the limited depth of the O-glycoproteome data available currently, and we expect the overlap to increase with deeper characterization of the two glycoproteomes. Thus, the identified O-Man glycosylation of nucleocytoplasmic proteins is likely to represent the equivalent to O-GlcNAcylation found in higher eukaryotes, and the discovery suggests that yeast possess a hitherto unidentified O-glycosylation machinery that operates in cytoplasmic, nuclear and mitochondrial compartments.

### Example 4

### O-Man Glycosites and Comparison to Mammalian O-GlcNAcylation Sites

We selected examples of orthologous proteins with known O-GlcNAcylation sites and identified O-Man glycosites for more detailed analysis. Although the existence of O-GlcNAc on histones was questioned in a recent study (Gagnon, Daou et al. 2015), O-GlcNAcylation is believed to be one of several post-translational modifications (PTMs) that constitute the histone code and regulate histone interactions with DNA and effector proteins, and this PTM has been found on histones H2A, H2B and H4 (Sakabe, Wang et al. 2010). It has further been demonstrated that O-GlcNAcylation of human histone H2B in response to glucose levels modulates the transcriptional response by promoting mono-ubiquitination of lysine residue 120 (Fujiki, Hashiba et al. 2011). A glucose-dependent response is also observed in yeast where the orthologous histone H2B.1 undergoes mono-ubiquitination at the conserved lysine residue, although the preceding step promoting this mono-ubiquitination has not been identified (Dong and Xu 2004). We found O-Man glycosites on the highly conserved yeast histone ortholog H2B.1 positioned virtually in the same region as O-GlcNAcylation on the human histone H2B (FIG. 2B). We found O-Man residues on the peptide TKYSSST¹²⁹, but could not assign the exact positions of the glycosites, however, an O-GlcNAc site has been identified in the highly conserved TSS sequon. Although, we in this first limited study did not identify O-Man glycosites on other histones, the results suggest that O-Man glycosylation is part of the histone code in yeast similar to O-GlcNAcylation in higher eukaryotes.

The plasma membrane H+-ATPase (PMA1) is O-GlcNAcylated in the cytosolic region in mammals (Trinidad, Barkan et al. 2012), and we identified O-Man glycosites on the yeast PMA1 ortholog that were conserved and overlapping with the known O-GlcNAcylation sites as well as phosphorylation sites (FIG. 2C). In particular, we found O-Man at Thr558, a conserved residue within the ATP binding motif of PMA1 (Davis, Smith et al. 1990), which is also O-GlcNAcylated in mammals (Trinidad, Barkan et al. 2012). The consequence of glycosylation at Thr558 is still unclear, but given that Thr558 is in immediate proximity to the ATP molecule, it is reasonable to predict that the O-glycan will have impact on ATP binding. PMA1 was further identified with O-Man modifications on the C-terminal regulatory domain where one site Ser899 has also been identified as being phosphorylated (FIG. 2C). The C-terminal region is a critical region that undergoes glucose-dependent phosphorylation leading to rapid PMA1 activation (Serrano 1983, Lecchi, Nelson et al. 2007). Upon glucose depletion, PMA1 is reversibly deactivated within minutes, suggesting a negative regulation driven by PTMs, although this mechanism is not fully understood.

In summary, the O-Man glycosites on nucleocytoplasmic proteins share the characteristic feature of overlap with phosphorylation sites and are positioned similarly to O-GlcNAc sites on evolutionary conserved proteins and sites. O-mannosylation of nucleocytoplasmic proteins in yeast was therefore considered likely to serve the equivalent functions as the O-GlcNAcylation process in higher eukaryotes. The nutrient sensing role of O-GlcNAcylation is likely to be mirrored by O-mannosylation in yeast with the difference being that GDP-Man, and not UDP-GlcNAc, will likely serve the role of donor substrate and key nutrient sensor in yeast. The biosynthesis of GDP-Man would thus link glucose and nucleotide metabolism in a network node capable of integrating nutrient signals that are ultimately relayed as nucleocytoplasmic O-Man glycosylation. The apparent shift to nutrient sensing by UDP-GlcNAc in higher eukaryotes from GDP-Man, may indicate a need to evolve a more complex nutrient sensor capable of incorporating glucose, nucleotide, amino acid, and fatty acid metabolism by flux through the hexosamine biosynthetic pathway. Yeast is the only eukaryotic cell type without identifiable orthologous OGT/OGA genes involved in O-GlcNAcylation, and the invention here provides an explanation since the enzymes required for transfer and removal of α-Mannose residues are likely to have entirely different structures and be encoded by non-homologous genes.

### Example 5

### O-Man Nucleocytoplasmic Glycosylation in Yeast is Dynamic and can be Modulated

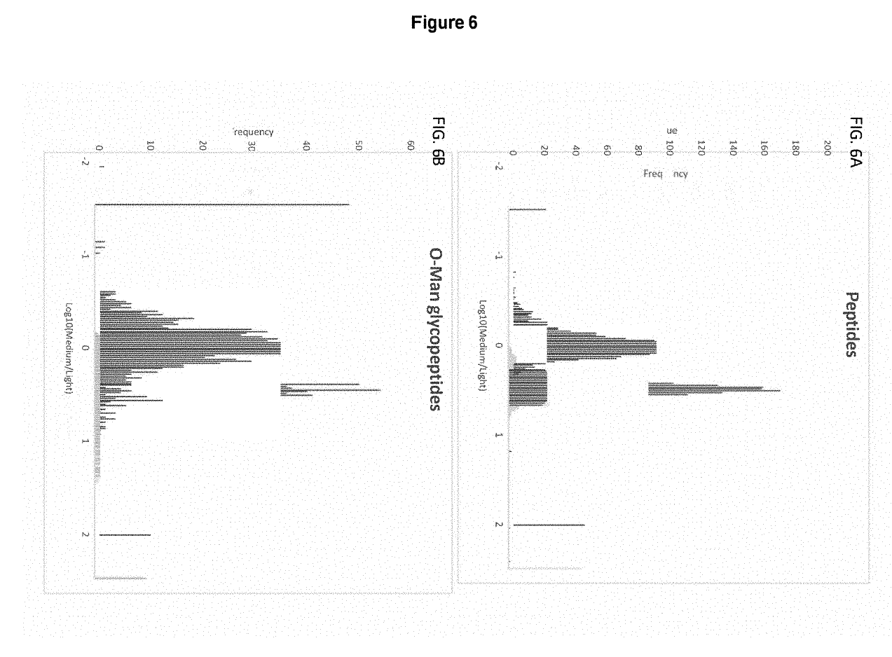

We probed the dynamics of nucleocytoplasmic O-Man glycosylations by subjecting WT yeast to a short burst of external stimuli. More specifically, a mid-log phase culture of WT Saccharomyces cerevisiae was divided in two and centrifuged (1000 g, 2 min). Growth media was discarded and one part of the pellet was resuspended in YPD growth media (described above) with nutrients (2% glucose) and the other part was resuspended in nutrient deprived growth media (0% glucose). Following 10 min stimuli, the yeast were lysed as described above, tryptic digests were differentially labelled with stable dimethyl isotopes and subjected to ConA LWAC, IEF and mass spectrometry. The tryptic digests of non-stimulated (2% glucose) yeast were labelled with “light” dimethyl reagent (resulting in N—(CH₃)₂) and the tryptic digests of stimulated yeast (0% glucose) were labelled with “medium” or deuterated (D) dimethyl reagent (resulting in N—(CD₃)₂). This labelling retains the chemico-physical properties of the peptides and O-Man glycopeptides but introduces a +4.0251 Da mass increment per label which is used to measure the relative change (quantity) between two samples. The differentially labelled peptides and glycopeptides were identified as described above and quantified using the “Precursor Ion Quantifier” node of Proteome Discoverer 1.4. In the ConA flow-through fraction, we identified >2000 peptides with quantification values. The quantification ratios (medium to light) of these non-glycosylated peptides were plotted on a log 10 scale and demonstrated a Gaussian distribution centred around 0, thereby showing that no significant changes had occurred at the proteome level (FIG. 6A) except for a few proteins that showed >100 fold up- or down regulation following exposure to nutrient deprivation. For the O-Man glycosylated peptides, we observed a similar distribution where the quantification ration (medium to light) of O-Man glycopeptides demonstrated a Gaussian distribution centered around 0, showing that no major changes had occurred on O-Man glycosylated peptides except for a few O-Man glycoproteins that showed >100 fold up- or down regulation following exposure to nutrient deprivation (FIG. 6B). Importantly, the significantly downregulated O-Man glycopeptides were all derived from proteins localized to nucleocytoplasmic compartments. ER/Golgi derived O-Man glycosylations were not identified among >100 fold downregulated O-Man glycosylations, thus showing that they are static with no significant changes in this experimental setup. This data therefore demonstrates that nucleocytoplasmic O-Man glycosylations are dynamic and respond to external stimuli by rapid modulation of nucleocytoplasmic O-Man glycosylations. This data further demonstrates that site-specific O-Man glycosylation of nucleocytoplasmic proteins is a dynamic process, and that a specific subset of O-Man glycosylation site are altered in response to specific external stimuli as demonstrated here for glucose. One of the proteins identified with significantly downregulated O-Man glycosylation was Ras-like protein 2 (RAS2), a modulator of adenylate cyclase activity and thus a master regulator of cyclic-AMP secondary messenger levels and protein kinase A (PKA) driven phosphorylation events.

### Example 6

### Identification and Characterization of an O-Man GDP-Man: Polypeptide Glycosyltransferase Activity in Yeast

In order to assay potential enzyme activity responsible for the O-Man glycosylation of nucleocytoplasmic protein, we designed a peptide substrate (LKNVPTPSPSPKPQH) based on identified glycoproteins covering the putative acceptor sites (Table 1).

### Methods

Wild-type S. cerevisiae were grown in YPD media (10 g/L of BactoYeast extract, 20 g/L of BactoPeptone and 20 g/L D-glucose) by shaking at 37° C. Cells were pelleted by centrifugation at 5000 rpm for 10 min and 5 ml packed cells were lysed in 10 ml ice-cold buffer containing 150 mM NaCl, 50 mM Tris pH 7.4 and complete protease inhibitors (Roche) with acid-washed glassbeads (0.4-0.8 mm in diameter) in a beadbeater (1 min pulse, 5 times). Soluble lysate was collected by ultracentrifugation at 50,000 g for 30 min using a SS-34 rotor. Enzymatic reactions containing 50 mM HEPES pH7.4, 1 mM DTT, 10 mM MgCl2, 0.01% Tween-20, 10 μM GDP-mannose* (mannose moiety is ¹⁴C isotope labeled; the specific activity is adjusted to 4000 cpm/nmol with non-labeled GDP-mannose) and 5 mM LKNVPTPSPSPKPQH were incubated with soluble lysate at room temperature for 3 hours before subjecting to weak ion exchange Dowex 1×8-100 resin for removal of un-reacted GDP-mannose. Samples were than either directed added to scintillation liquid and subject to scintillation counting or injected onto C18 RP-HPLC for further separations. Fractions were then collected and subjected to scintillation counting for demonstration of incorporation of ¹⁴C-Man into peptide substrates.

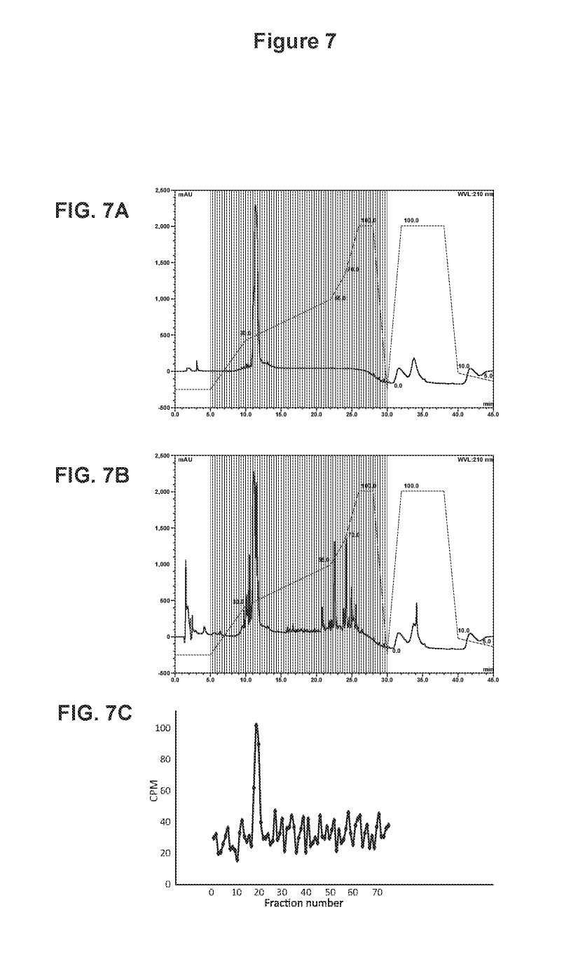

Enzymatic incorporation of ¹⁴C labeled mannose transferred from GDP-mannose into the peptide substrate (LKNVPTPSPSPKPQH) was demonstrated by both direct scintillation counting of total enzyme reactions after removal of unreacted GDP-mannose by Dowex chromatography as well as by C18 RP-HPLC analysis of fractions containing the peptide and O-Man glycopeptide (FIG. 7). Using S. cerevisiae soluble lysate and an enzymatic assay involving a synthetic peptide, enzymatic O-mannosylation of peptide substrates derived from nucleocytoplasmic proteins undergong O-mannosylation in yeast was demonstrated.

TABLE 1
S. cerevisiae

| | Systematic | | |
|---|---|---|---|
| UniProt ID | ID | Gene | Sites |
| D6VTK4 | YFL026W | STE2 | 363; 366; 368 |
| O14455 | YPL249C-A | RPL36B | 5 |
| O14467 | YOR298C-A | MBF1 | 63; 69 |
| O14467 | YOR298C-A | MBF1 | 50; 51; 53 |
| P00330 | YOL086C | ADH1 | 41; 46 |
| P00358 | YJR009C | TDH2 | 199; 201; 207; 208; 209 |
| P00358 | YJR009C | TDH2 | 251; 252 |
| P00358 | YJR009C | TDH2 | 139; 140 |
| P00359 | YGR192C | TDH3 | 251; 252 |
| P00359 | YGR192C | TDH3 | 139; 140 |
| P00359 | YGR192C | TDH3 | 199; 201; 207; 208; 209 |
| P00360 | YJL052W | TDH1 | 199; 201; 207; 208; 209 |
| P00431 | YKR066C | CCP1 | 67; 68; 69; 76 |
| P00549 | YAL038W | CDC19 | 492; 494 |
| P00924 | YGR254W | ENO1 | 188; 196 |
| P00925 | YHR174W | ENO2 | 188; 196 |
| P02293 | YDR224C | HTB1 | 123; 126; 127; 128; 129 |
| P02293 | YDR224C | HTB1 | 100 |
| P02557 | YFL037W | TUB2 | 143; 145; 149; 153 |
| P02994 | YBR118W | TEF2 | 267 |
| P02994 | YPR080W | TEF1 | 267 |
| P04037 | YGL187C | COX4 | 33; 45 |
| P05030 | YGL008C | PMA1 | 899 |
| P05030 | YGL008C | PMA1 | 558; 568 |
| P05030 | YGL008C | PMA1 | 459; 464 |
| P06169 | YLR044C | PDC1 | 514; 515; 522 |
| P06367 | YCR031C | RPS14A | 77 |
| P06367 | YCR031C | RPS14A | 22; 26; 31; 34 |
| P06367 | YCR031C | RPS14A | 119; 123; 125; 126 |
| P06738 | YPR160W | GPH1 | 31 |
| P06738 | YPR160W | GPH1 | 77; 78 |
| P06774 | YGL237C | HAP2 | 255; 256; 257 |
| P06776 | YOR360C | PDE2 | 139; 142; 147; 150; 159 |
| P07246 | YMR083W | ADH3 | 68; 73 |
| P07280 | YOL121C | RPS19A | 97; 99 |
| P08018 | YJL128C | PBS2 | 151; 156; 161; 164; 166; 168; 170; 172; 174 |
| P08153 | YDR146C | SWI5 | 114; 118; 119 |
| P08153 | YDR146C | SWI5 | 505; 510 |
| P08153 | YDR146C | SWI5 | 488; 490; 492 |
| P0C0X0 | YLR264W | RPS28B | 5; 8 |
| P0C2I5 | | | 28; 36; 38; 40 |
| P0C2I8 | | | 28; 36; 38; 40 |
| P14540 | YKL060C | FBA1 | 56; 62; 63 |
| P14540 | YKL060C | FBA1 | 11; 24 |
| P14540 | YKL060C | FBA1 | 347; 349; 352 |
| P14907 | YJL041W | NSP1 | 580; 581; 583; 584; 589 |
| P14907 | YJL041W | NSP1 | 298; 299; 304 |
| P14922 | YBR112C | CYC8 | 715; 720; 725; 728 |
| P15891 | YCR088W | ABP1 | 163; 165; 167; 169; 174 |
| P19097 | YPL231W | FAS2 | 31 |
| P19097 | YPL231W | FAS2 | 67; 73 |
| P20048 | YMR013C | SEC59 | 315 |
| P21147 | YGL055W | OLE1 | 506 |
| P21192 | YLR131C | ACE2 | 501 |
| P21306 | YPL271W | ATP15 | 39; 41; 46 |
| P21306 | YPL271W | ATP15 | 52; 55; 58; 61 |
| P21375 | YJR051W | OSM1 | 251; 252 |
| P21576 | YKR001C | VPS1 | 559; 565; 568; 569; 570 |
| P22133 | YOL126C | MDH2 | 357 |
| P22133 | YOL126C | MDH2 | 372 |
| P22211 | YNL183C | NPR1 | 326; 328; 330; 336; 338; 339; 347; 349 |
| P23292 | YNL154C | YCK2 | 69; 70 |
| P23585 | YMR011W | HXT2 | 29; 32; 36 |
| P23644 | YMR203W | TOM40 | 15; 20; 23 |
| P23797 | YMR281W | GPI12 | 205 |
| P24784 | YPL119C | DBP1 | 375 |
| P24813 | YDR423C | CAD1 | 337; 339; 346; 347; 350 |
| P24813 | YDR423C | CAD1 | 312; 313 |
| P24813 | YDR423C | CAD1 | 320; 323; 330; 332 |
| P25297 | YML123C | PHO84 | 465; 466; 467; 477 |
| P25297 | YML123C | PHO84 | 500; 504 |
| P25297 | YML123C | PHO84 | 577; 578; 579; 581 |
| P25297 | YML123C | PHO84 | 22 |
| P25357 | YCR033W | SNT1 | 64; 69; 74; 75; 84 |
| P25358 | YCR034W | ELO2 | 334; 336; 338 |
| P25573 | YCL044C | MGR1 | 353 |
| P26786 | YOR096W | RPS7A | 133 |
| P27895 | YEL061C | CIN8 | 984; 985; 993 |
| P28272 | YKL216W | URA1 | 230 |
| P29509 | YDR353W | TRR1 | 165; 174 |
| P29509 | YDR353W | TRR1 | 237; 242; 251; 254 |
| P32047 | YNL074C | MLF3 | 79; 87; 88 |
| P32047 | YNL074C | MLF3 | 407; 408; 411; 414; 415; 416; 418; 420; 421 |
| P32338 | YGR044C | RME1 | 135; 137; 138; 141; 143 |
| P32338 | YGR044C | RME1 | 224 |
| P32338 | YGR044C | RME1 | 109; 110 |
| P32338 | YGR044C | RME1 | 72 |
| P32338 | YGR044C | RME1 | 153 |
| P32338 | YGR044C | RME1 | 138; 141; 143 |
| P32338 | YGR044C | RME1 | 166; 167; 169; 173 |
| P32342 | YKL122C | SRP21 | 77; 79 |
| P32342 | YKL122C | SRP21 | 69 |
| P32457 | YLR314C | CDC3 | 501; 503; 509; 513 |
| P32486 | YPR159W | KRE6 | 116; 117; 122 |
| P32599 | YDR129C | SAC6 | 102; 103; 107; 108; 111 |
| P32614 | YEL047C | FRD1 | 31 |
| P32790 | YBL007C | SLA1 | 454; 467 |
| P32839 | YDR375C | BCS1 | 227; 232 |
| P32904 | YHR147C | MRPL6 | 48; 49; 53; 57 |
| P33336 | YGR143W | SKN1 | 196; 200; 202 |
| P33894 | YOR219C | STE13 | 77 |
| P35197 | YDL226C | GCS1 | 168; 170; 174; 175 |
| P35732 | YKL054C | DEF1 | 86; 101 |
| P35997 | YKL156W | RPS27A | 11; 14 |
| P36024 | YKR072C | SIS2 | 76; 77; 82; 84 |
| P36060 | YKL150W | MCR1 | 59 |
| P36093 | YKL043W | PHD1 | 138; 147; 150; 151 |
| P36093 | YKL043W | PHD1 | 322 |
| P36112 | YKR016W | MIC60 | 273; 275 |
| P36112 | YKR016W | MIC60 | 304; 310 |
| P37267 | YGR174C | CBP4 | 80; 85 |
| P38073 | YBR033W | EDS1 | 665 |
| P38166 | YBL102W | SFT2 | 37; 40; 41; 45; 48 |
| P38236 | YBR057C | MUM2 | 162; 170 |
| P38272 | YBR130C | SHE3 | 392; 393; 394; 396; 398; 404 |
| P38314 | YBR214W | SDS24 | 85; 90; 92; 94; 98 |
| P38330 | | | 127; 132; 134; 137 |
| P38631 | YLR342W | FKS1 | 1857; 1860; 1864; 1866; 1868 |
| P38749 | YHL009C | YAP3 | 133; 135; 146 |
| P38797 | YHR076W | PTC7 | 38; 42; 45; 51; 52; 54; 56; 60 |
| P38797 | YHR076W | PTC7 | 173; 174; 182 |
| P38881 | YHR195W | NVJ1 | 65; 75 |
| P38968 | YDL195W | SEC31 | 814; 815; 816; 817; 822 |
| P38968 | YDL195W | SEC31 | 974; 975; 977; 980 |
| P38968 | YDL195W | SEC31 | 940; 948 |
| P38968 | YDL195W | SEC31 | 900; 908; 911 |
| P38969 | YOR266W | PNT1 | 408; 414; 419 |
| P38972 | YGR061C | ADE6 | 333; 338; 341; 348; 350 |
| P39013 | YNL084C | END3 | 17 |
| P39523 | YMR124W | EPO1 | 370 |
| P39726 | YAL044C | GCV3 | 38 |
| P39928 | YIL147C | SLN1 | 786; 791 |
| P39935 | YGR162W | TIF4631 | 2; 5; 9; 11; 13 |
| P39935 | YGR162W | TIF4631 | 908; 911; 912 |
| P39940 | YER125W | RSP5 | 191; 192; 195; 197; 198; 201; 203; 205; 206; 207 |
| P39952 | YER154W | OXA1 | 33; 36 |
| P39960 | YER155C | BEM2 | 92; 93; 94; 95; 96; 97; 106; 108; 110 |
| P40005 | YEL003W | GIM4 | 89; 93 |
| P40049 | | | 59; 63; 67; 71 |
| P40087 | YER143W | DDI1 | 378; 379; 383; 384; 386 |
| P40087 | YER143W | DDI1 | 368; 373; 375 |
| P40107 | YGL225W | VRG4 | 6; 16; 20; 24 |
| P40159 | | | 47; 48; 53; 60 |
| P40356 | YGL025C | PGD1 | 134; 135; 137; 140; 141; 144; 148; 149; 151; 152; 153; 155 |
| P40463 | YIL135C | VHS2 | 108; 117 |
| P40529 | YIL044C | AGE2 | 195; 196; 197; 199; 200; 202; 203; 204; 206; 207; 213 |
| P40564 | YIR004W | DJP1 | 321; 322 |
| P41821 | YNL291C | MID1 | 451; 452; 453; 454; 455 |
| P42839 | YNL321W | VNX1 | 26; 28; 29; 30; 32; 33 |
| P42845 | YNL309W | STB1 | 129; 131 |
| P42939 | YGR206W | MVB12 | 91 |
| P43574 | YFL021W | GAT1 | 399 |
| P43594 | YFR011C | MIC19 | 61; 67 |
| P43594 | YFR011C | MIC19 | 108 |
| P43638 | YJL042W | MHP1 | 209; 213; 218 |
| P47068 | YJL020C | BBC1 | 608; 609; 612; 613; 620; 621; 624 |
| P47116 | YJR059W | PTK2 | 722; 726; 727; 729; 730; 732; 737 |
| P47116 | YJR059W | PTK2 | 729; 730; 732; 737 |
| P47143 | YJR105W | ADO1 | 258; 264; 268; 269 |
| P48015 | YDR019C | GCV1 | 58; 66; 71 |
| P48559 | YNL304W | YPT11 | 8; 13; 14; 16; 19; 21; 23 |
| P48562 | YNL298W | CLA4 | 311; 314; 316; 322 |
| P49723 | YGR180C | RNR4 | 332; 334; 336 |
| P50276 | YGR055W | MUP1 | 6; 9 |
| P50896 | YDR505C | PSP1 | 282; 283; 286 |
| P53075 | YGL228W | SHE10 | 290 |
| P53075 | YGL228W | SHE10 | 196; 202; 204 |
| P53159 | YGL075C | MPS2 | 373; 382 |
| P53172 | YGL056C | SDS23 | 400; 402; 404; 405; 409; 410; 413; 416; 417; 420; 422; 424; 425 |
| P53220 | YGR033C | TIM21 | 24; 28; 29; 32; 34; 35 |
| P53297 | YGR178C | PBP1 | 230; 231; 238; 239 |
| P53319 | YGR256W | GND2 | 479; 486; 487; 488 |
| P53599 | YNR031C | SSK2 | 1254; 1257; 1258; 1260; 1263 |
| P53885 | YNL173C | MDG1 | 306; 308; 310; 313 |
| P53901 | YNL152W | INN1 | 384; 385; 386; 388; 392 |
| P53946 | YNL059C | ARP5 | 58; 60 |
| P54785 | YMR070W | MOT3 | 314; 315; 326; 327; 332; 339; 340 |
| Q00246 | YKR055W | RHO4 | 264; 268; 269; 270; 273; 274; 276; 278; 280 |
| Q03063 | YPL049C | DIG1 | 142; 148; 151 |
| Q03088 | YPL032C | SVL3 | 689; 694; 702; 709 |
| Q03088 | YPL032C | SVL3 | 662; 665; 667 |
| Q03104 | YML128C | MSC1 | 52; 54; 60 |
| Q03104 | YML128C | MSC1 | 52; 54 |
| Q03104 | YML128C | MSC1 | 415; 417; 419; 420 |
| Q03104 | YML128C | MSC1 | 52; 54; 60; 63 |
| Q03104 | YML128C | MSC1 | 419; 420 |
| Q03104 | YML128C | MSC1 | 415; 417; 419; 420; 429 |
| Q03281 | YDR458C | HEH2 | 411 |
| Q03281 | YDR458C | HEH2 | 607; 608 |
| Q03640 | YML072C | TCB3 | 1279; 1285; 1291; 1295 |
| Q03667 | YMR002W | MIX17 | 23; 24; 41; 50 |
| Q03707 | YML034W | SRC1 | 642; 644; 652 |
| Q03772 | YDR163W | CWC15 | 21; 23 |
| Q04439 | YMR109W | MYO5 | 1054; 1058; 1066 |
| Q04439 | YMR109W | MYO5 | 992; 1001; 1005 |
| Q04947 | YDR233C | RTN1 | 246 |
| Q04947 | YDR233C | RTN1 | 197; 206 |
| Q04958 | YML059C | NTE1 | 21; 24; 25; 28; 29; 32; 33 |
| Q04958 | YML059C | NTE1 | 25; 28; 29; 32; 33 |
| Q04958 | YML059C | NTE1 | 577; 582 |
| Q04958 | YML059C | NTE1 | 584; 589 |
| Q04964 | YML058W | SML1 | 25; 27 |
| Q05050 | YMR031C | EIS1 | 50; 51; 56 |
| Q06011 | YLR390W | ECM19 | 102; 105; 108 |
| Q06179 | YLR454W | FMP27 | 730; 734; 737 |
| Q06315 | YLR187W | SKG3 | 977; 982 |
| Q06608 | | | 101; 103; 105; 106 |
| Q06616 | | | 168; 169; 170; 175; 179 |
| Q06681 | YDR326C | YSP2 | 1375; 1379; 1382 |
| Q06681 | YDR326C | YSP2 | 1333 |
| Q07362 | YDL053C | PBP4 | 28; 32 |
| Q07549 | YDL123W | SNA4 | 113; 116 |
| Q07793 | | | 28; 36; 38; 40 |
| Q08001 | | | 44; 45; 46; 52 |
| Q08001 | | | 66; 67; 74; 78 |
| Q08300 | | | 140; 141; 148; 151 |
| Q08300 | | | 169; 170 |
| Q08300 | | | 148; 151; 158; 161 |
| Q12019 | YLR106C | MDN1 | 193; 200; 204; 207 |
| Q12199 | YPR040W | TIP41 | 39; 40; 43; 44; 46; 50; 55 |
| Q12199 | YPR040W | TIP41 | 16 |
| Q12273 | | | 28; 36; 38; 40 |
| Q12276 | YOR227W | HER1 | 50; 51; 56 |
| Q12328 | YDL217C | TIM22 | 4; 12 |
| Q12363 | YOR230W | WTM1 | 37; 39; 41 |
| Q12427 | YDR169C | STB3 | 334; 336; 337; 340; 341 |
| Q12443 | YDL204W | RTN2 | 318 |
| Q12443 | YDL204W | RTN2 | 362; 363; 368; 370 |
| Q12515 | YDL173W | PAR32 | 141; 145; 146; 147; 148 |
| Q12515 | YDL173W | PAR32 | 85 |
| Q12515 | YDL173W | PAR32 | 138; 141; 145; 146; 147; 148 |
| Q12522 | YPR016C | TIF6 | 74; 76; 77 |
| Q3E823 | YPL189C-A | COA2 | 37; 40; 42 |

S. pombe

| UniProt ID | Systematic ID | Gene | Sites |
|---|---|---|---|
| A6X980 | SPAC1250.07 | sfc7 | 101; 103; 104; 105; 108; 110; 118 |
| O13601 | SPBC11B10.05c | rsp1 | 103; 104; 108 |
| O13702 | SPAC13F5.03c | gld1 | 442; 446 |
| O13735 | SPAC15A10.16 | bud6 | 171; 175; 180 |
| O13736 | SPAC16E8.01 | shd1 | 927; 928; 929; 944 |
| O13900 | SPAC22A12.09c | sap114 | 468; 470; 475; 477 |
| O13962 | SPAC24C9.02c | cyt2 | 34; 39; 43; 47 |
| O14007 | SPAC29A4.04c | cbf5 | 383; 388 |
| O14015 | SPAC29A4.12c | mug108 | 6; 7; 8; 11; 14 |
| O14015 | SPAC29A4.12c | mug108 | 6; 7; 8; 11; 14; 23; 25 |
| O14018 | SPAC29A4.15 | srs1 | 395; 396; 400 |
| O14033 | SPAC29B12.12 | SPAC29B12.12 | 91 |
| O14069 | SPAC1687.06c | rp144 | 46; 60 |
| O14160 | SPAC4A8.08c | vrs2 | 269 |
| O42644 | SPAC10F6.03c | cts1 | 81; 83; 89; 90 |
| O42663 | SPAC27D7.09c | SPAC27D7.09c | 226; 227 |
| O42663 | SPAC27D7.09c | SPAC27D7.09c | 222; 226; 227 |
| O42663 | SPAC27D7.09c | SPAC27D7.09c | 227 |
| O42663 | SPAC27D7.09c | SPAC27D7.09c | 222; 226; 227; 242; 246 |
| O42665 | SPAC27D7.11c | SPAC27D7.11c | 296; 298; 302; 304; 306 |
| O42665 | SPAC27D7.11c | SPAC27D7.11c | 302; 304; 306 |
| O42665 | SPAC27D7.11c | SPAC27D7.11c | 292; 293; 296; 298; 302; 304; 306 |
| O42665 | SPAC27D7.11c | SPAC27D7.11c | 304; 306 |
| O42665 | SPAC27D7.11c | SPAC27D7.11c | 306 |
| O42665 | SPAC27D7.11c | SPAC27D7.11c | 298; 302; 304; 306 |
| O42665 | SPAC27D7.11c | SPAC27D7.11c | 265; 266; 267; 272; 274; 276; 277; 282; 283 |
| O42667 | SPAC27D7.13c | ssm4 | 295; 298; 301 |
| O42700 | SPCC1020.06c | tal1 | 33; 34; 37; 43 |
| O42917 | SPBC16A3.11 | eso1 | 623 |
| O42976 | SPBC20F10.07 | SPBC20F10.07 | 741 |
| O42976 | SPBC20F10.07 | SPBC20F10.07 | 761 |
| O42980 | SPBC17D11.01 | nep1 | 371; 379; 383; 388 |
| O43002 | SPBC2G2.03c | sbh1 | 53; 54; 56 |
| O43024 | SPBC354.10 | def1 | 417; 429 |
| O43024 | SPBC354.10 | def1 | 695; 703; 705; 707; 712 |
| O43024 | SPBC354.10 | def1 | 510; 519; 524 |
| O59667 | SPBC29A3.02c | his7 | 321; 323 |
| O59733 | SPBC3E7.13c | syf2 | 120 |
| O59824 | SPCC965.04c | yme1 | 120; 122; 126; 128; 130; 131; 132; 133; 136; 137; 138; 140 |
| O60057 | SPBC56F2.06 | mug147 | 295 |
| O60057 | SPBC56F2.06 | mug147 | 295; 302 |
| O60069 | SPBC13G1.07 | swf1 | 308 |
| O60073 | SPBC13G1.11 | ykt6 | 148 |
| O60081 | SPCC1494.07 | SPCC1494.07 | 903; 904; 909 |
| O60101 | SPBC14F5.04c | pgk1 | 373; 375 |
| O74417 | SPCC14G10.04 | SPCC14G10.04 | 167; 174; 175 |
| O74423 | SPCC162.07 | ent1 | 510; 518 |
| O74423 | SPCC162.07 | ent1 | 494; 499; 510; 518 |
| O74454 | SPCC16C4.09 | sts5 | 67; 75; 76 |
| O74718 | SPCC584.04 | sup35 | 77; 86; 89 |
| O74718 | SPCC584.04 | sup35 | 77; 86; 89; 95 |
| O74840 | SPCC1235.03 | cue2 | 368; 370; 377 |
| O74864 | SPCC18.14c | rpp0 | 24; 34; 35; 40 |
| O74895 | SPCC576.11 | rpl15 | 183; 188 |
| O74989 | SPCC338.12 | pbi2 | 73 |
| O74989 | SPCC338.12 | pbi2 | 36; 39 |
| O74989 | SPCC338.12 | pbi2 | 5 |
| O74989 | SPCC338.12 | pbi2 | 57; 73 |
| O94248 | SPCC737.08 | SPCC737.08 | 3903; 3905; 3910; 3912; 3916; 3924 |
| O94259 | SPBP8B7.10c | utp16 | 62; 67; 68; 69; 71; 73 |
| O94274 | SPBP8B7.26 | SPBP8B7.26 | 24; 25 |
| O94274 | SPBP8B7.26 | SPBP8B7.26 | 57 |
| O94305 | SPCC5E4.05c | mgl1 | 341; 342; 344; 345; 346; 350; 354; 355; 356 |
| O94358 | SPBC428.10 | SPBC428.10 | 646; 647; 649; 650 |
| O94385 | SPBC29A10.07 | pom152 | 144; 145 |
| O94399 | SPCC126.06 | twf1 | 306; 307; 312; 313; 314; 315 |
| O94428 | SPBC660.09 | mug168 | 79; 81; 82; 84; 88 |
| O94428 | SPBC660.09 | mug168 | 54; 55; 58; 64; 66 |
| O94428 | SPBC660.09 | mug168 | 58; 64; 66; 69 |
| O94428 | SPBC660.09 | mug168 | 64; 66; 69 |
| O94432 | SPBC660.15 | msi2 | 333 |
| O94700 | SPBC83.17 | SPBC83.17 | 72; 73; 74 |
| O94733 | SPCC191.01 | SPCC191.01 | 7; 8 |
| P0CT53 | SPCC794.09c | tef101 | 289; 296 |
| P19117 | SPAC23C11.05 | ipp1 | 29; 30 |
| P28758 | SPAC821.10c | sod1 | 55; 59; 60 |
| P36580 | SPBC19C2.07 | fba1 | 312 |
| P36584 | SPAPB8E5.06c | rpl302 | 328 |
| P40370 | SPBC1815.01 | eno101 | 189; 192; 195 |
| P40372 | SPAC17A5.03 | rpl301 | 328 |
| P78812 | SPBC660.16 | SPBC660.16 | 143 |
| P79058 | SPAC10F6.16 | mug134 | 89; 92 |
| P79058 | SPAC10F6.16 | mug134 | 99; 102; 108; 111 |
| P79082 | SPBC27.06c | mgr2 | 81; 83 |
| P87148 | SPBC25H2.06c | hrf1 | 11; 17 |
| P87175 | SPBC3D6.10 | apn2 | 355; 357; 359; 363; 365; 367; 368; 370; 371; 373; 375 |
| P87216 | SPAC10F6.06 | vip1 | 189; 193; 194 |
| P87216 | SPAC10F6.06 | vip1 | 169; 175; 176; 177; 180 |
| P87216 | SPAC10F6.06 | vip1 | 141; 144 |
| P87216 | SPAC10F6.06 | vip1 | 115; 118 |
| P87216 | SPAC10F6.06 | vip1 | 175; 176; 177; 180; 181 |
| P87216 | SPAC10F6.06 | vip1 | 230; 232; 235; 236; 237; 239; 247; 250; 251; 253 |
| P87216 | SPAC10F6.06 | vip1 | 101; 105; 109 |
| P87216 | SPAC10F6.06 | vip1 | 122; 128; 132; 133 |
| P87216 | SPAC10F6.06 | vip1 | 2; 8; 11; 15 |
| P87216 | SPAC10F6.06 | vip1 | 156 |
| Q09719 | SPAC31A2.07c | dbp10 | 133; 134; 138; 140; 146 |
| Q09748 | SPBC12C2.08 | dnm1 | 291 |
| Q09811 | SPAC2G11.12 | rqh1 | 1261; 1263; 1265; 1267; 1269 |
| Q10104 | SPAC18G6.04c | shm2 | 89 |
| Q10234 | SPAC4G9.17c | mrp5 | 186; 190; 191 |
| Q10475 | SPAC17C9.03 | tif471 | 318; 329 |
| Q92371 | SPAC4F10.14c | btf3 | 75; 78; 79; 84; 85 |
| Q9HFE8 | SPBP16F5.03c | tra1 | 615; 616; 622; 626; 627; 629; 630; 633 |
| Q9P3A9 | SPAC1565.05 | utp8 | 303 |
| Q9P6M1 | SPAC688.04c | gst3 | 228; 232 |
| Q9P6M1 | SPAC688.04c | gst3 | 228; 232; 240 |
| Q9P7L2 | SPBC21C3.11 | ubx4 | 241; 243; 245; 248; 251; 253 |
| Q9P7U1 | SPAC977.15 | SPAC977.15 | 194; 198; 202; 204; 208 |
| Q9URZ2 | SPAC869.09 | SPAC869.09 | 37; 39; 42; 43; 45; 46 |
| Q9URZ2 | SPAC869.09 | SPAC869.09 | 16; 21 |
| Q9US22 | SPAC1783.08c | rp11502 | 183; 188 |
| Q9UT74 | SPAC343.16 | lys2 | 677; 678; 680; 682; 683; 684 |
| Q9UTC5 | SPAC227.14 | yfh7 | 43; 44 |
| Q9UTR5 | SPAC1006.06 | rgf2 | 43; 44; 46; 48; 50; 52; 55; 56 |
| Q9UUA1 | SPCC23B6.01c | SPCC23B6.01c | 400; 403; 408; 409; 414 |
| Q9UUE1 | SPBC17G9.11c | pyr1 | 13; 14; 16; 20 |
| Q9UUG0 | SPAC926.09c | fas1 | 2055; 2057 |
| Q9Y7K2 | SPBC216.07c | tor2 | 1327 |
| Q9Y7R8 | SPCC306.08c | mdh1 | 146; 151; 152 |
| Q9Y7U0 | SPCC63.14 | eis1 | 439; 440; 448 |
| Q9Y7U0 | SPCC63.14 | eis1 | 227 |
| Q9Y7U0 | SPCC63.14 | eis1 | 1160; 1166; 1172 |
| Q9Y7U0 | SPCC63.14 | eis1 | 596; 597; 601; 602 |
| Q9Y7U0 | SPCC63.14 | eis1 | 992; 996; 997 |
| Q9Y7U0 | SPCC63.14 | eis1 | 116; 117; 118; 121 |
| Q9Y7U0 | SPCC63.14 | eis1 | 245; 251; 252 |
| Q9Y7X6 | SPBC365.12c | ish1 | 234 |
| Q9Y7X6 | SPBC365.12c | ish1 | 234; 243 |
| Q9Y7X6 | SPBC365.12c | ish1 | 437; 439; 443 |
| Q9Y7X6 | SPBC365.12c | ish1 | 393; 395; 396; 404; 406; 407 |
| Q9Y7X6 | SPBC365.12c | ish1 | 675 |
| Q9Y7X6 | SPBC365.12c | ish1 | 254 |
| Q9Y7X6 | SPBC365.12c | ish1 | 389; 393; 395; 396; 404; 406; 407 |
| Q9Y7X6 | SPBC365.12c | ish1 | 126; 130; 132; 134 |
| Q9Y7X6 | SPBC365.12c | ish1 | 663; 664; 667; 668; 675 |
| Q9Y7X6 | SPBC365.12c | ish1 | 569; 575; 577; 583 |
| Q9Y7X6 | SPBC365.12c | ish1 | 667; 668; 675 |
| Q9Y7X6 | SPBC365.12c | ish1 | 320 |
| Q9Y7X6 | SPBC365.12c | ish1 | 658; 660; 663; 664; 667; 668; 675 |
| Q9Y7X6 | SPBC365.12c | ish1 | 303 |
| Q9Y7X6 | SPBC365.12c | ish1 | 384; 389; 393; 395; 396; 404; 406; 407 |
| Q9Y7X6 | SPBC365.12c | ish1 | 418; 421; 422; 424 |
| Q9Y7X6 | SPBC365.12c | ish1 | 418; 421; 422; 424; 437; 439 |
| Q9Y7X6 | SPBC365.12c | ish1 | 21; 25; 26 |
| Q9Y7X6 | SPBC365.12c | ish1 | 86; 88; 89; 95 |
| Q9Y7X6 | SPBC365.12c | ish1 | 437; 439 |
| Q9Y7X6 | SPBC365.12c | ish1 | 658; 660 |
| Q9Y7X6 | SPBC365.12c | ish1 | 225; 226; 234 |
| Q9Y7X6 | SPBC365.12c | ish1 | 160; 163; 167 |
| Q9Y7X6 | SPBC365.12c | ish1 | 268; 269 |
| Q9Y7X6 | SPBC365.12c | ish1 | 337 |
| Q9Y7X6 | SPBC365.12c | ish1 | 664; 667; 668; 675 |
| Q9Y7X6 | SPBC365.12c | ish1 | 556; 560; 563 |
| Q9Y7X6 | SPBC365.12c | ish1 | 393; 395; 396 |
| Q9Y7X6 | SPBC365.12c | ish1 | 452; 456 |
| Q9Y7X6 | SPBC365.12c | ish1 | 37; 43; 48 |
| Q9Y7X6 | SPBC365.12c | ish1 | 197; 207; 208; 211; 212 |
| Q9Y7X6 | SPBC365.12c | ish1 | 389; 393; 395; 396 |
| Q9Y7X6 | SPBC365.12c | ish1 | 389; 393; 395; 396; 404 |
| Q9Y7X6 | SPBC365.12c | ish1 | 389; 393; 395; 396; 404; 406 |
| Q9Y7X6 | SPBC365.12c | ish1 | 634; 640 |
| Q9Y7X6 | SPBC365.12c | ish1 | 367; 370; 378; 381; 384 |
| Q9Y7X6 | SPBC365.12c | ish1 | 437; 439; 443; 447 |
| Q9Y7X6 | SPBC365.12c | ish1 | 74; 80 |
| Q9Y7X6 | SPBC365.12c | ish1 | 418; 421; 422; 424; 437; 439; 443 |

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary to employ concepts of the various patents, application and publications to provide yet further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

Applicants incorporate by reference herein in its entirety the following article (and all related supplements, supporting information, and data depositions: Discovery of a nucleocytoplasmic O-mannose glycoproteome in yeast; by Adnan Halima, Ida Signe Bohse Larsen, Patrick Neubert, Hiren Jitendra Joshia, Bent Larsen Petersen, Sergey Y. Vakhrushev, Sabine Strahl, and Henrik Clausen. Proc Natl Acad Sci USA. 2015 Dec. 22; 112(51):15648-53. This article is available at the following world-wide-web address: pnas.org/cgi/doi/10.1073/pnas.1511743112, and the supplement for this article is also incorporated herein by reference in its entirety, and is available at the following world-wide-web address: pnas.org/content/112/51/15648?tab=ds.

Data Deposition:

The mass spectrometry proteomics data related to the abovementioned PNAS manuscript have been deposited in the ProteomeXchange Consortium via the PRoteomics IDEntifications (PRIDE) Partner Repository (accession no. PXD002924), and this data is available at the world-wide-web addresss: proteomecentral.proteomexchange.org/cgi/GetDataset?ID=PXD002924). The content of this data deposition is incorporated herein by reference in its entirety.

Supporting Information:

This manuscript also contains supporting information that is available online at the world wide web address: pnas.org/lookup/suppl/doi:10.1073/pnas.1511743112/-/DCSupplemental. The content of this supporting information is incorporated herein by reference in its entirety.

### REFERENCES

Alfaro, J. F., C. X. Gong, M. E. Monroe, J. T. Aldrich, T. R. Clauss, S. O. Purvine, Z. Wang, D. G. Camp, 2nd, J. Shabanowitz, P. Stanley, G. W. Hart, D. F. Hunt, F. Yang and R. D. Smith (2012). “Tandem mass spectrometry identifies many mouse brain O-GlcNAcylated proteins including EGF domain-specific O-GlcNAc transferase targets.” Proc Natl Acad Sci USA 109(19): 7280-7285.
Arroyo, J., J. Hutzler, C. Bermejo, E. Ragni, J. Garcia-Cantalejo, P. Botias, H. Piberger, A. Schott, A. B. Sanz and S. Strahl (2011). “Functional and genomic analyses of blocked protein O-mannosylation in baker's yeast.” Mol Microbiol 79(6): 1529-1546.
Bodenmiller, B., D. Campbell, B. Gerrits, H. Lam, M. Jovanovic, P. Picotti, R. Schlapbach and R. Aebersold (2008). “PhosphoPep—a database of protein phosphorylation sites in model organisms.” Nat Biotechnol 26(12): 1339-1340.
Bond, M. R. and J. A. Hanover (2015). “A little sugar goes a long way: the cell biology of O-GlcNAc.” J Cell Biol 208(7): 869-880.
Cao, W., J. Cao, J. Huang, J. Yao, G. Yan, H. Xu and P. Yang (2013). “Discovery and confirmation of O-GlcNAcylated proteins in rat liver mitochondria by combination of mass spectrometry and immunological methods.” PLoS One 8(10): e76399.
Chi, A., C. Huttenhower, L. Y. Geer, J. J. Coon, J. E. Syka, D. L. Bai, J. Shabanowitz, D. J. Burke, O. G. Troyanskaya and D. F. Hunt (2007). “Analysis of phosphorylation sites on proteins from Saccharomyces cerevisiae by electron transfer dissociation (ETD) mass spectrometry.” Proc Natl Acad Sci USA 104(7): 2193-2198.
Ciucanu, I. and C. E. Costello (2003). “Elimination of oxidative degradation during the per-O-methylation of carbohydrates.” J Am Chem Soc 125(52): 16213-16219.
Davis, C. B., K. E. Smith, B. N. Campbell, Jr. and G. G. Hammes (1990). “The ATP binding site of the yeast plasma membrane proton-translocating ATPase.” J Biol Chem 265(3): 1300-1305.
Dong, D. L. and G. W. Hart (1994). “Purification and characterization of an O-GlcNAc selective N-acetyl-beta-D-glucosaminidase from rat spleen cytosol.” J Biol Chem 269(30): 19321-19330.
Dong, L. and C. W. Xu (2004). “Carbohydrates induce mono-ubiquitination of H2B in yeast.” J Biol Chem 279(3): 1577-1580.
Endo, T. (2015). “Glycobiology of alpha-dystroglycan and muscular dystrophy.” J Biochem 157(1): 1-12.
Fosset, M., L. W. Muir, L. D. Nielsen and E. H. Fischer (1971). “Purification and properties of yeast glycogen phosphorylase a and b.” Biochemistry 10(22): 4105-4113.
Fujiki, R., W. Hashiba, H. Sekine, A. Yokoyama, T. Chikanishi, S. Ito, Y. Imai, J. Kim, H. H. He, K. Igarashi, J. Kanno, F. Ohtake, H. Kitagawa, R. G. Roeder, M. Brown and S. Kato (2011). “GlcNAcylation of histone H2B facilitates its monoubiquitination.” Nature 480(7378): 557-560.
Gagnon, J., S. Daou, N. Zamorano, N. V. Iannantuono, I. Hammond-Martel, N. Mashtalir, E. Bonneil, H. Wurtele, P. Thibault and B. Affar el (2015). “Undetectable histone O-GlcNAcylation in mammalian cells.” Epigenetics 10(8): 677-691.
Gentzsch, M. and W. Tanner (1996). “The PMT gene family: protein O-glycosylation in Saccharomyces cerevisiae is vital.” EMBO J 15(21): 5752-5759.
Gnad, F., J. Gunawardena and M. Mann (2011). “PHOSIDA 2011: the posttranslational modification database.” Nucleic Acids Res 39 (Database issue): D253-260.
Haltiwanger, R. S., M. A. Blomberg and G. W. Hart (1992). “Glycosylation of nuclear and cytoplasmic proteins. Purification and characterization of a uridine diphospho-N-acetylglucosamine:polypeptide beta-N-acetylglucosaminyltransferase.” J Biol Chem 267(13): 9005-9013.
Hardiville, S. and G. W. Hart (2014). “Nutrient regulation of signaling, transcription, and cell physiology by O-GlcNAcylation.” Cell Metab 20(2): 208-213.
Hart, G. W., M. P. Housley and C. Slawson (2007). “Cycling of O-linked beta-N-acetylglucosamine on nucleocytoplasmic proteins.” Nature 446(7139): 1017-1022.
Hart, G. W., C. Slawson, G. Ramirez-Correa and O. Lagerlof (2011). “Cross talk between O-GlcNAcylation and phosphorylation: roles in signaling, transcription, and chronic disease.” Annu Rev Biochem 80: 825-858.
Heese-Peck, A., R. N. Cole, O. N. Borkhsenious, G. W. Hart and N. V. Raikhel (1995). “Plant nuclear pore complex proteins are modified by novel oligosaccharides with terminal N-acetylglucosamine.” Plant Cell 7(9): 1459-1471.
Holt, G. D. and G. W. Hart (1986). “The subcellular distribution of terminal N-acetylglucosamine moieties. Localization of a novel protein-saccharide linkage, O-linked GlcNAc.” J Biol Chem 261(17): 8049-8057.
Hornbeck, P. V., J. M. Kornhauser, S. Tkachev, B. Zhang, E. Skrzypek, B. Murray, V. Latham and M. Sullivan (2012). “PhosphoSitePlus: a comprehensive resource for investigating the structure and function of experimentally determined post-translational modifications in man and mouse.” Nucleic Acids Res 40 (Database issue): D261-270.
Hu, P., S. Shimoji and G. W. Hart (2010). “Site-specific interplay between O-GlcNAcylation and phosphorylation in cellular regulation.” FEBS Lett 584(12): 2526-2538.
Lecchi, S., C. J. Nelson, K. E. Allen, D. L. Swaney, K. L. Thompson, J. J. Coon, M. R. Sussman and C. W. Slayman (2007). “Tandem phosphorylation of Ser-911 and Thr-912 at the C terminus of yeast plasma membrane H+-ATPase leads to glucose-dependent activation.” J Biol Chem 282(49): 35471-35481.
Lin, K., P. K. Hwang and R. J. Fletterick (1995). “Mechanism of regulation in yeast glycogen phosphorylase.” J Biol Chem 270(45): 26833-26839.
Loibl, M. and S. Strahl (2013). “Protein O-mannosylation: what we have learned from baker's yeast.” Biochim Biophys Acta 1833(11): 2438-2446.
Lommel, M. and S. Strahl (2009). “Protein O-mannosylation: conserved from bacteria to humans.” Glycobiology 19(8): 816-828.
Sadowski, I., B. J. Breitkreutz, C. Stark, T. C. Su, M. Dahabieh, S. Raithatha, W. Bernhard, R. Oughtred, K. Dolinski, K. Barreto and M. Tyers (2013). “The PhosphoGRID Saccharomyces cerevisiae protein phosphorylation site database: version 2.0 update.” Database (Oxford) 2013: bat026.
Sakabe, K., Z. Wang and G. W. Hart (2010). “Beta-N-acetylglucosamine (O-GlcNAc) is part of the histone code.” Proc Natl Acad Sci USA 107(46): 19915-19920.
Serrano, R. (1983). “In vivo glucose activation of the yeast plasma membrane ATPase.” FEBS Lett 156(1): 11-14.
Torres, C. R. and G. W. Hart (1984). “Topography and polypeptide distribution of terminal N-acetylglucosamine residues on the surfaces of intact lymphocytes. Evidence for O-linked GlcNAc.” J Biol Chem 259(5): 3308-3317.
Trinidad, J. C., D. T. Barkan, B. F. Gulledge, A. Thalhammer, A. Sali, R. Schoepfer and A. L. Burlingame (2012). “Global identification and characterization of both O-GlcNAcylation and phosphorylation at the murine synapse.” Mol Cell Proteomics 11(8): 215-229.
Vakhrushev, S. Y., C. Steentoft, M. B. Vester-Christensen, E. P. Bennett, H. Clausen and S. B. Levery (2013). “Enhanced mass spectrometric mapping of the human GalNAc-type O-glycoproteome with SimpleCells.” Mol Cell Proteomics 12(4): 932-944.
Vester-Christensen, M. B., A. Halim, H. J. Joshi, C. Steentoft, E. P. Bennett, S. B. Levery, S. Y. Vakhrushev and H. Clausen (2013). “Mining the O-mannose glycoproteome reveals cadherins as major O-mannosylated glycoproteins.” Proc Natl Acad Sci USA 110(52): 21018-21023.
Vizcaino, J. A., E. W. Deutsch, R. Wang, A. Csordas, F. Reisinger, D. Rios, J. A. Dianes, Z. Sun, T. Farrah, N. Bandeira, P. A. Binz, I. Xenarios, M. Eisenacher, G. Mayer, L. Gatto, A. Campos, R. J. Chalkley, H. J. Kraus, J. P. Albar, S. Martinez-Bartolome, R. Apweiler, G. S. Omenn, L. Martens, A. R. Jones and H. Hermjakob (2014). “ProteomeXchange provides globally coordinated proteomics data submission and dissemination.” Nat Biotechnol 32(3): 223-226.
Wang, J., M. Torii, H. Liu, G. W. Hart and Z. Z. Hu (2011). “dbOGAP—an integrated bioinformatics resource for protein O-GlcNAcylation.” BMC Bioinformatics 12: 91.

1-27. (canceled)

28. A method of producing a polypeptide comprising expressing a recombinant polypeptide in a yeast cell comprising a modulated nucleocytoplasmic alpha-mannosyltransferase enzyme activity.

29. The method of claim 28, wherein the yeast cell exhibits improved bioprocessing due to the modulated nucleocytoplasmic alpha-mannosyltransferase activity.

30. The method of claim 28, wherein the nucleocytoplasmic alpha-mannosyltransferase activity is modulated by modulating the cells' nutrient supply.

31. The method of claim 28, wherein the cell is a yeast cell selected from Saccharomyces cerevisiae or Schizosaccharomyces pombe.

32. The method of claim 28, wherein the nucleocytoplasmic alpha-mannosyltransferase activity is modulated by one or more of:
A. increasing the cell's available pool of GDP-Man;
B. decreasing the cell's available pool of GDP-Man
C. modulating the transport of GDP-Man to the nucleocytoplasmic compartment of the cell;
D. contacting the cell with an inhibitor or an activator of a nucleocytoplasmic alpha-mannosyltransferase enzyme;
E. overexpressing a nucleocytoplasmic alpha-mannosyltransferase enzyme; and/or
F. knocking down or knocking out a native nucleocytoplasmic alpha-mannosyltransferase enzyme.

33.-37. (canceled)

38. The method of claim 28, wherein the method comprises modulating nucleocytoplasmic O-Man glycosylation found on a polypeptide selected from the group consisting of one or more (i) carbohydrate transporter; (ii) enzyme involved in the catalysis pentose phosphate pathway; (iii) enzyme involved in the catalysis of the citric acid cycle; (iv) enzyme involved in the catalysis of the fermentation; (v) enzyme catalyzing glycolysis, gluconeogenesis, fermentation, the pentose phosphate pathway the citric acid cycle, or combinations thereof; (vi) enzyme involved in the catalysis of oxidative phosphorylation; (vii) polypeptide comprised in a plasma membrane ATPase protein complex, a vacuolar ATPase protein complex, or a combination thereof; (viii) enzyme involved in the catalysis of nitrogen metabolism; (ix) enzyme involved in the catalysis of lipid metabolism; (x) carbohydrate sensors; (x) enzyme involved in the catalysis of the biosynthesis of glycosyl-phosphatidylinositol (GPI) membrane anchors; (xii) enzyme that control levels of secondary messenger molecule cyclic adenosine monophosphate (cAMP); (xiii) enzyme involved in the catalysis of glycolysis, gluconeogenesis; and (xiv) enzyme involved in the catalysis of glycogen metabolism, trehalose metabolism.

39. The method of claim 38, wherein the polypeptide is selected from one or more of AGT1, GAL2, HXT1-17, MAL61, HXK1, HXK2, GLK1, PGI1, PFK1, PFK2, FBP1, FBA1, TPI1, GPD1, GPD2, GUT2, GPP1, GPP2, GUT1, THD1-3, PGK1, GPM1, ENO1, ENO2, PYK1, PYK2, PDA1, PDB1, LAT1, LPD1, PDX1, PCK1, PYC1, PYC2, TAL1, CIT1-3, ACO1, IDH1, IDH2, IDP1, IDP2, KGD1, KGD2, ACS1, ACS2, LPD1, LSC1, LSC2, SDH1-4, FUM1, MDH1-3, ICL1, MLS1, MLS2, PDC1, PDC5, ACS1, ACS2, PDC6, ALD4, ALD6, ADH1-5, SDHA, SDHB, SDHC, SDHD, ISP, COB, CYT1, COR1, QCR2, QCR6, QCR7, QCR8, QCR9, QCR10, COX1-4, COX5A, COX5B, COX6A, COX6B, COX7A, COX7C, COX10, COX11, COX15, ATP1-9, ATP14-21, PMA1, VMA1-8, VMA10, VMA13, STV, GSY1, GSY2, GPH1, GLT1, GDH1-3, GLN1, GPI1-3, GPI15, GPI12, YJR013w, GPI10, SMP3, GPI13, GPI17, GPI11, GAA1, GPI8, GPI15, GPI17, YLR459w, SNF3, RGT2, RAS1, RAS2, CDC25, IRA1, IRA2, and CYR1.

40. The method of claim 39, wherein the cell exhibits modulated metabolism, carbon metabolism, growth on non-fermentable compounds, levels of downstream carbon metabolites, ethanol production; nicotinamide adenine dinucleotide [NAD(H)] levels, nicotinamide adenine dinucleotide phosphate [NAD(H)] levels, flavin adenine dinucleotide [FAD(H2)] levels, energy homeostatis, proton gradients, adenosine triphosphate (ATP) levels, intracellular and/or vacuolar pH levels, levels of storage carbohydrates, downstream carbon metabolites, nitrogen metabolism, downstream nitrogen metabolites, GPI-anchor synthesis, GPI-anchored protein synthesis, and/or downstream cellular signalling.

41.-76. (canceled)

77. The method of claim 28, wherein the cell exhibits improved production of the exogenous polypeptide as compared to a corresponding yeast cell lacking any such modulation of its nucleocytoplasmic alpha-mannosyltransferase enzyme activity.

78. The method of claim 77, wherein the improved production comprises an increase in production yield of the polypeptide.

79.-104. (canceled)

105. A composition comprising a recombinant polypeptide with one or more mutation in its amino acid sequence as compared to its native polypeptide sequence, wherein the mutation modulates the O-mannosylation of the polypeptide.

106. The composition of claim 105, wherein the mutation results in decreased O-mannosylation of the polypeptide.

107. The composition of claim 105, wherein the mutation results in increased O-mannosylation of the polypeptide.

108. The composition of claim 105, comprising one or more mutation of an O-Man glycosite.

109. The composition of claim 108, wherein the mutated O-Man glycosite is any one of the O-Man glycosites identified in Table 1.

110. A yeast cell comprising one or more gene encoding one or more polypeptide that has been mutated to produce proteins with one or more amino acid substitutions that eliminates one or more O-Man glycosylation sites.

111. (canceled)
